(19) United States
(12) Patent Application Publication
(10) Pub. No.: US 2025/0059609 A1
(43) Pub. Date: Feb. 20, 2025

(54) BIOMARKERS FOR THE IDENTIFICATION OF AGGRESSIVE PROSTATE CANCER AND METHODS OF TREATMENT THEREOF

(71) Applicant: DUKE UNIVERSITY, Durham, NC (US)

(72) Inventors: Qing CHENG, Durham, NC (US); Jiaoti HUANG, Durham, NC (US)

(21) Appl. No.: 18/721,782

(22) PCT Filed: Dec. 20, 2022
(86) PCT No.: PCT/US2022/082008

§ 371 Date: Jun. 19, 2024

Related U.S. Application Data
(60) Provisional application No. 63/292,190, filed on Dec. 21, 2021.

(51) Int. Cl. C12Q 1/6886

(52) U.S. Cl. CPC C12Q 1/6886; C12Q 2600/118; C12Q 2600/158

(57) ABSTRACT

The present disclosure describes, in part, biomarkers for the identification of aggressive prostate cancer by determining a castration-resistant prostate cancer (CRPC) evolutionary signature of prostate cells, and methods of use thereof.

### CROSS-REFERENCE TO RELATED APPLICATIONS

This application claims the benefit of U.S. Provisional Patent Application No. 63/292,190, filed Dec. 21, 2021, the contents of which is hereby incorporate by reference.

### FEDERAL FUNDING LEGEND

This invention was made with Government support under Federal Grant nos. CA205001 and CA200853 awarded by the National Institutes of Health (NIH). The Federal Government has certain rights to this invention.

### BACKGROUND

The vast majority of prostate cancer (PCa) cases are classified as adenocarcinoma with glandular formation and luminal differentiation including the expression of androgen receptor (AR) and prostate specific antigen (PSA). Hormonal therapy, by inhibiting AR signaling, controls PCa initially but leads to the development of castration-resistant prostate cancer (CRPC). Most CRPCs are still adenocarcinoma histologically (CRPC-adeno); however, a significant proportion of the clinical CRPC cases belongs to a histologic variant form of carcinoma called small cell neuroendocrine carcinoma (SCNC), which is highly aggressive. In comparison to adenocarcinoma, SCNC is composed of tumor cells that have lost luminal differentiation and acquired neuroendocrine differentiation (NED) through lineage plasticity, rather than developing resistant mutations. There is a need for better ways of identifying and treating these types of cancers.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

Provided herein according to some aspects is a method of determining the risk of disease progression of prostate cancer in a subject. The method may include steps of: (i) obtaining a biological sample from the subject, said sample comprising prostate cancer cells; and (ii) determining a castration-resistant prostate cancer (CRPC) evolutionary signature of the prostate cancer cells to generate a score, wherein a sample having a high score as compared to a control is indicative of aggressive prostate cancer.

In some aspects, the signature comprises a plurality of genes and the determining comprises measuring the expression levels of the genes. In some aspects, the measuring is carried out with quantitative PCR such as rtPCR.

In some aspects, the signature comprises from 3, 5, 8 or 10 to 20, 25, 30, 35, 40, 45, or 51 of the genes listed in Table 1. In some aspects, the signature comprises from 3, 5, 8 or 10 to 20, 25, 30, 35, 40, 45, or 51 of the genes listed as CRPCsig51_old as provided in Table 1. In some aspects, the signature comprises from 3, 5, 8 or 10 to 20, 25, 30, 35, 40, 45, or 51 of the genes listed as CRPCsig51_new as provided in Table 1. In some aspects, the signature comprises from 3, 4, 5, or 6, to 8, 9, 10, 11, or 12 of the genes listed in Table 4.

In some aspects, the score is determined by detecting upregulation of expression of genes of the CRPC evolutionary signature. In some aspects, the score comprises a Youden's index, or a score based on maximum specificity and sensitivity.

In some aspects, the subject has an early-stage prostate cancer diagnosis, and may be determined to be of intermediate risk.

Also provided is a method of detecting and treating an aggressive prostate cancer in a subject. The method may include: (i) obtaining a biological sample from the subject, said sample comprising prostate cancer cells; (ii) determining a castration-resistant prostate cancer (CRPC) evolutionary signature in the sample to generate a score, in which samples from the subject have a higher score as compared to a control, which higher score is indicative of the aggressive prostate cancer; and then (iii) administering a prostate cancer treatment to the subject determined to have aggressive prostate cancer.

In some aspects, the prostate cancer treatment comprises one or more of surgery (e.g., prostatectomy), radiation, and focal treatment (e.g., heat, cold, or laser treatment).

In some aspects, the signature comprises a plurality of genes and the determining comprises measuring the expression levels of the genes. In some aspects, the measuring is carried out with quantitative PCR such as rtPCR.

In some aspects, the signature comprises from 3, 5, 8 or 10 to 20, 25, 30, 35, 40, 45, or 51 of the genes listed in Table 1. In some aspects, the signature comprises from 3, 5, 8 or 10 to 20, 25, 30, 35, 40, 45, or 51 of the genes listed as CRPCsig51_old as provided in Table 1. In some aspects, the signature comprises from 3, 5, 8 or 10 to 20, 25, 30, 35, 40, 45, or 51 of the genes listed as CRPCsig51_new as provided in Table 1. In some aspects, the signature comprises from 3, 4, 5, or 6, to 8, 9, 10, 11, or 12 of the genes listed in Table 4.

In some aspects, the score is determined by a Youden's index, or maximum specificity and sensitivity.

In some aspects, the subject has an early-stage prostate cancer diagnosis, and may be determined to be of intermediate risk.

Further provided is the use of a genetic signature for the determination of a disease state in a subject suffering from prostate cancer, the genetic signature comprising from 3, 5, 8 or 10 to 20, 25, 30, 35, 40, 45, or 51 of the genes listed in Table 1. In some aspects, the genetic signature comprises from 3, 5, 8 or 10 to 20, 25, 30, 35, 40, 45, or 51 of the genes listed as CRPCsig51_old in Table 1. In some aspects, the genetic signature comprises from 3, 5, 8 or 10 to 20, 25, 30, 35, 40, 45, or 51 of the genes listed as CRPCsig51_new in Table 1. In some aspects, the genetic signature comprises from 3, 4, 5, or 6, to 8, 9, 10, 11, or 12 of the genes listed in Table 4.

The present disclosure is based, in part, on the hypothesis by the inventors that the fact that PCa initially responds to hormonal therapy but develops resistance eventually suggests that sub-clones may exist in the primary tumor with different degrees of AR dependency and sensitivity to hormonal therapy.

Based on this hypothesis, the inventors have developed a fresh prostate tissue procurement protocol that analyzes purified epithelial cells isolated from PCa by performing single-cell RNA sequencing (scRNA-seq) to characterize a large number of individual tumor cells. The results show that primary PCa comprise a non-uniform distribution of genetically distinct tumor-cell subpopulations, and the temporal changes in genetic landscape under therapeutic pressure facilitates treatment resistance and disease progression.

Accordingly, one aspect of the present disclosure provides a genetic signature for the determination of a disease state in a subject suffering from, or at risk of suffering from, prostate cancer, the genetic signature comprising, consisting of, or consisting essentially of one or more genes provided in Table 1. In one embodiment, the genetic signature comprises one or more genes listed as CRPCsig51_old as provided in Table 1. In another embodiment, the genetic signature comprises one or more genes listed as CRPCsig51_new as provided in Table 1.

Another aspect of the present disclosure provides a method of determining the risk of disease progression in a subject suffering from, or at risk of suffering from, prostate cancer, the method comprising, consisting of, or consisting essentially of (i) obtaining a biological sample from the subject; and (ii) determining the CRPC evolutionary signature in the sample to generate a score, in which samples having a high score as compared to a control is indicative of aggressive prostate cancer.

Another aspect of the present disclosure provides a method of determining the risk of disease progression in a subject suffering from, or at risk of suffering from, prostate cancer, the method comprising, consisting of, or consisting essentially of (i) obtaining a biological sample from the subject; (ii) determining the CRPC evolutionary signature in the sample to generate a score, in which samples having a high score as compared to a control is indicative of aggressive prostate cancer and (iii) administering early and/or aggressive prostate cancer treatments to the subject if the score is higher than that of the control.

Another aspect of the present disclosure provides a method of determining whether early and/or aggressive prostate cancer treatments should be administered to a subject, the method comprising, consisting of, or consisting essentially of (i) obtaining a biological sample from the subject; (ii) determining the CRPC evolutionary signature in the sample to generate a score, in which samples having a high score as compared to a control is indicative of aggressive prostate cancer and (iii) administering early and/or aggressive prostate cancer treatments to the subject if the score is higher than that of the control.

In one embodiment, the genetic signature comprises one or more genes listed as CRPCsig51_old as provided in Table 1. In another embodiment, the genetic signature comprises one or more genes listed as CRPCsig51_new as provided in Table 1.

Another aspect provides an assay for determining the CRPCsig51 genetic signature in a subject, the method comprising, consisting of, or consisting essentially of (i) obtaining a biological sample from the patient; (ii) determining a genetic signature from the biological sample; and (iii) providing an output comprising the genetic signature.

Other aspects provide for kits for determining a genetic signature in a subject.

Another aspect of the present disclosure provides all that is described and illustrated herein.

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more embodiments, in which:

FIG. 1A-FIG. 1G present data showing intra-tumoral heterogeneity in primary PCa and CRPC in accordance with one embodiment of the present disclosure.

FIG. 1A. Histology and IHC images. Panel (a): Representative image of primary PCa (GS 4+3). Panel (b): Locally recurrent CRPC-adeno. Panel (c): Metastatic CRPC (mCRPC) to pelvic side wall. Tumor shows classic features of CRPC-adeno. Panel (d): Locally recurrent PCa shows classic architectural and cytologic features of SCNC. Panel (e): Positive cytoplasmic staining of SCNC cells in panel (d) for the expression of a NE marker synaptophysin (SYP). Panel (f): Positive nuclear staining of SCNC cells in panel (d) for the expression of a NE marker TTF-1. Scale bars in each panel are equal to 50 μm.

FIG. 1B. UMAP projection of expression profiles of 24,385 cells isolated from primary PCa and CRPC/SCNC samples. Dots represent single cells, grouped by cell clusters.

FIG. 1C. Feature plots of cell type and lineage markers. Shading represents expression level, from no expression (light gray) to high level expression (dark gray).

FIG. 1D. UMAP projection of cells shaded by sample.

FIG. 1E. UMAP projection of cells shaded by lineage subtypes. Cell clusters were classified as basal, NE (in primary PCa), PSA-low and PSA-high luminal (in primary PCa), mCRPC with PSA-high, local CRPC with AR-high, SCNC and lymphocyte.

FIG. 1F. UMAP view of tumor-adjacent tissue cell distribution among the cell clusters.

FIG. 1G. Distribution of cells isolated from tumor-adjacent tissue, PCa and CRPC/SCNC samples among luminal clusters.

FIG. 2A-FIG. 2E present data showing subtype specific oncogenic signaling in accordance with one embodiment of the present disclosure.

FIG. 2A. UMAP view of cellular pathway signaling, shaded by the quartiles of signature score.

FIG. 2B. UMAP view of cellular gene set signature scores. Shading represents signature score level, from no expression (light gray) to high level expression (dark gray).

FIG. 2C. Antibody-based IHC detection of ERG rearrangement. Panel (a): PCal sample. Panel (b): PCa2 sample. Panel (c) PCa3 sample. Scale bars in each panel are equal to 50 μm.

FIG. 2D. PCa markers defined subtype differences in tumor (both PCa and CRPC cells) and tumor-adjacent tissue. Violin plot visualized cellular gene expression within a cluster. Dot represents the median score level.

FIG. 2E. Lineage and PCa marker defined subtype differences among the cells isolated from primary PCa samples.

FIG. 2F. RB inactivation status among the cells isolated from primary PCa samples. Violin plot visualized the cellular signature score within a cluster. Dot represents the median score level.

FIG. 3 presents data showing the characterization of evolutionary trajectories and CRPC-like cells in primary PCa samples in accordance with one embodiment of the present disclosure.

FIG. 3A. Alignment of cells along trifurcating trajectories of CRPC progression. The trajectory analysis was performed using non-basal clusters, and only cells isolated from CRPC samples were analyzed. Dots represent single cells. Solid lines represent distinct cell trajectories defined by single-cell transcriptomes. Shading represents individual cell cluster. Arrow line represents trajectory direction.

FIG. 3B. Bifurcating trajectories of CRPC progression through AR-dependent and independent mechanisms. Both PCa and CRPC cells from non-basal clusters were included in this analysis.

FIG. 3C. UMAP projection of cells in C2, C8 and C12 clusters isolated from both primary PCa and CRPC/SCNC samples.

FIG. 3D. UMAP projection of cells in C2, C8 and C12 clusters isolated from primary PCa samples.

FIG. 3E. UMAP projection of cells in C2, C8 and C12 clusters, isolated from primary PCa samples.

FIG. 3F. Distribution of NE cells on the trajectory of SCNC progression (Trajectory 1), shaded by samples. Non-basal cells from both primary PCa and CRPC/SCNC samples were analyzed. Cells from mCRPC (C2) and CRPC-adeno (C12) clusters were excluded. We determined the NE cells by visualizing which C8 cells (FIG. 12A) were isolated from primary PCa samples. Arrows indicate the NE cells.

FIG. 3G. Distribution of CRPC-like cells on the trajectory of mCRPC progression (Trajectory 2), shaded by samples. Non-basal cells from both primary PCa and CRPC/SCNC samples were analyzed. Cells from SCNC (C8) and CRPC-adeno (C12) clusters were excluded. We determined the CRPC-like cells by visualizing which C2 or C12 cells (FIG. 12B) were isolated from primary PCa samples. Arrows indicate the CRPC-like cells.

FIG. 4A-FIG. 4B present data showing the prognosis of the CRPC-like cells related CRPC/SCNC evolutionary signature (CRPCsig51) in accordance with one embodiment of the present disclosure.

FIG. 4A. Association between CRPCsig51 signature and biochemical recurrence (BCR)-free survival. Kaplan-Meier estimates of BCR-free survival in 140 PCa cases obtained from GSE21034 (p<0.001, HR=22.10, 95% CI: 8.70, 56.13). By assessing the diagnostic ability of CRPCsig51 on local and distant metastasis (N1 or M1, AJCC stage IV), Youden's index was selected as the cut-point for CRPCsig51-high vs. CRPCsig51-low samples. p-values were calculated using Mantel-cox test.

FIG. 4B. CRPCsig51-high was significantly associated with higher risk of PCa disease progression (both clinical and biochemical recurrence). Kaplan-Meier estimates of progression-free survival in 485 PCa cases obtained from TCGA (p<0.001; HR=3.10; 95% CI: 2.10, 4.58).

FIG. 5A-FIG. 5C present data showing the distribution of CRPC-like cells in primary PCa and CRPC/mCRPC samples in accordance with one embodiment of the present disclosure.

FIG. 5A. Visualization of CRPC-like and NE cells in hormone sensitive PCa samples. Representative IHC images of paired hormone sensitive PCa slides staining with: Panel (a) TOP2A, Panel (b) CHGA, Panel (c) NUSAP1, Panel (d) CHGA, Panel (e) TOP2A, and Panel (f) CHGA. CRPC-like cells are negative for NE marker, while NE cell is negative for CRPC-like markers. Arrows indicate the positive staining cells. Scale bars in each panel are equal to 25 μm.

FIG. 5B. CRPC-like cells are highly enriched in CRPC and mCRPC samples. Representative IHC images of hormone sensitive PCa staining with (a) TOP2A (b) NUSAP1 (c) PHGR1; CRPC staining (d) TOP2A (e) NUSAP1 (f) PHGR1; and mCRPC staining with (g) TOP2A (h) NUSAP1 (i) PHGR1. Scale bars in each panel are equal to 25 μm.

FIG. 5C. Visualization of PCa samples with enriched CRPC-like cells in an independent dataset of 685 samples, using CRPCsig51 score, SYP expression and the expression of 8 CRPC-like cell markers. The combined dataset of PCa 685 samples was developed using 9 datasets (E-TABM-26, GSE17951, GSF2443, GSE25136, GSF32269, GSF32448, GSF3325, GSE6956, and GSE8218) that gene expression profiles were measured using Affymetrix U133A or U133 Plus 2.0 expression array.

FIG. 6A-FIG. 6B present data showing barcode Rank Plot of single cell libraries in accordance with one embodiment of the present disclosure.

FIG. 6A. Barcode Rank Plots of 13 single cell libraries were generated from primary PCa and benign, local recurrent CRPC, and mCRPC samples. The y-axis is the number of UMI counts mapped to each barcode and the x-axis is the number of barcodes below that value. A steep drop-off is indicative of good separation between the cell-associated barcodes and the barcodes associated with empty partitions, suggesting each of the 13 libraries passed QC.

FIG. 6B. Barcode Rank Plot of Combined cell library data. Barcode Rank Plot was generated by Cell Ranger for the visualization of single cell library quality.

FIG. 7A-FIG. 7F present data showing pre-clustering single cell RNA (scRNA) sequencing data in accordance with one embodiment of the present disclosure.

FIG. 7A. PCA plot of raw data after CellRanger normalization, shaded by batches. Multiple single cell libraries were combined using CellRanger through deep normalization, in order to avoid the batch effect introduced by sequencing depth. Primary sources of heterogeneity in the dataset (raw data) were visualized using PCA plot, colored by batches.

FIG. 7B. PCA plot after Seurat linear dimensional reduction, shaded by batches. The CellRanger normalized sequencing data was further processed by Seurat for removing unwanted cells (if they expressed less than 500 genes or expressed over 8000 genes, and if the percentage of mitochondrial genes was greater than 10% per cell). Primary sources of heterogeneity in the dataset (scared expression data) before clustering were visualized using PCA plot, colored by batches.

FIG. 7C. Primary sources of heterogeneity in the dataset measured by Seurat V2.

FIG. 7D. tSNE projection of expression profiles of 24,385 single epithelial cells isolated from primary PCa and CRPC/SCNC samples, using Seurat V2. Dots represent single cells, grouped by Seurat defined cell clusters.

FIG. 7E. Primary sources of heterogeneity in the dataset measured by Seurat V3.

FIG. 7F. UMAP projection of expression profiles of 24,385 single epithelial cells isolated from primary PCa and CRPC/SCNC samples using Seurat V3. Dots represent single cells, grouped by Seurat defined cell clusters.

FIG. 8A-FIG. 8C present data showing characterization of cell clusters in accordance with one embodiment of the present disclosure.

FIG. 8A. Heatmap of top 10 marks from each cluster.

FIG. 8B. Feature plots of 8 epithelial cell markers. Shading represents expression level, from no expression (light gray) to high level expression (dark gray).

FIG. 8C. IHC images of CHGA (NE cell marker) and cluster C12 specific markers (CD68, HLA-DR and Lysozyme). Arrows point to the cells co-stained with NE and immune related markers. The scale bars in each panel are equal to 25 μm.

FIG. 9A-FIG. 9F present data showing distribution of cells isolated from different tissue in accordance with one embodiment of the present disclosure.

FIG. 9A. tSNE projection of expression profiles of cells isolated from primary PCa samples (both tumor and benign), grouped by Seurat defined cell clusters.

FIG. 9B. tSNE projection of expression profiles of cells isolated from primary PCa tumor samples, grouped by Seurat defined cell clusters.

FIG. 9C. tSNE projection of expression profiles of cells isolated from primary PCa benign tissue, grouped by Seurat defined cell clusters.

FIG. 9D. tSNE projection of expression profiles of cells isolated from CRPC samples (both tumor and benign of local recurrent SCNC), grouped by Seurat defined cell clusters.

FIG. 9E. tSNE projection of expression profiles of cells isolated from CRPC tumor samples, grouped by Seurat defined cell clusters.

FIG. 9F. tSNE projection of expression profiles of cells isolated from benign of local recurrent SCN, grouped by Seurat defined cell clusters.

FIG. 10A-FIG. 10B present data showing the characterization of cell clusters using lineage markers and pathway signatures in accordance with one embodiment of the present disclosure.

FIG. 10A. Characterization of cell clusters using lineage markers. Bar represents average marker expression of the cells within same cluster. Lineage class markers were selected for measuring SCNC (NCAM1, SYN1), luminal (TMPRSS2, CDH1), and basal (TP63). Bar represents average marker expression of cells within same cluster.

FIG. 10B. Heatmap of MSigDB hallmark and oncogenic pathway signaling across 24,385 single epithelial cells. Cells were grouped by Seurat defined cell clusters, and clusters were further grouped by lineage subtypes. First principal component (PC1) of the gene set was used to assign each cell a signaling score, shaded by score level. MSigDB hallmark gene sets were used to access well-defined biological states or processes, and de-regulated cellular pathways in cancer cells was defined using MSigDB oncogenic gene sets.

FIG. 11A-FIG. 11H present data showing the CRPC trajectories defined by non-basal CRPC cells in accordance with one embodiment of the present disclosure.

FIG. 11A. Trajectory of SCNC progression, grouped by cell clusters. Only non-basal CRPC cells were analyzed. Cells from mCRPC clusters (C3) and AR-high CRPC cluster (C10) were excluded.

FIG. 11B. Trajectory of PSA-high mCRPC progression, grouped by cell clusters. Only non-basal CRPC cells were analyzed. Cells from SCNC clusters (C8) and AR-high CRPC cluster (C10) were excluded.

FIG. 11C. Trajectory of AR-high CRPC progression, grouped by cell clusters. Only non-basal CRPC cells were analyzed. Cells from SCNC clusters (C8) and mCRPC cluster (C10) were excluded.

FIG. 11D-FIG. 11H. Monocle ordered cells on Trajectory 1A, Trajectory 1B, Trajectory 2, Trajectory 3A, and Trajectory 3B, grouped by pseudotime. Only non-basal CRPC cells were analyzed. Cells from mCRPC clusters (C3) and AR-high CRPC cluster (C10) were excluded. Light gray dots represent cells excluded from the analysis.

FIG. 12A-FIG. 12G present data showing the CRPC trajectories defined by both non-basal PCa and CRPC cells in accordance with one embodiment of the present disclosure.

FIG. 12A. Trajectory of SCNC progression, grouped by cell clusters. Both non-basal PCa and CRPC cells were analyzed. Cells from mCRPC clusters (C3) and AR-high CRPC cluster (C10) were excluded.

FIG. 12B. Trajectory of PSA-high mCRPC progression, grouped by cell clusters. Both non-basal PCa and CRPC cells were analyzed. Cells from SCNC clusters (C8) and AR-high CRPC cluster (C10) were excluded.

FIG. 12C. Trajectory of AR-high CRPC progression, grouped by cell clusters. Both non-basal PCa and CRPC cells were analyzed. Cells from SCNC clusters (C8) and mCRPC cluster (C3) were excluded.

FIG. 12D. Monocle ordered cells on Trajectory 1, grouped by pseudotime. Both non-basal PCa and CRPC cells were analyzed. Cells from mCRPC clusters (C3) and AR-high CRPC cluster (C10) were excluded. Light gray dots represent cells excluded from the analysis.

FIG. 12E. Monocle ordered cells on Trajectory 2, grouped by pseudotime. Both non-basal PCa and CRPC cells were analyzed. Cells from SCNC clusters (C8) and AR-high CRPC cluster (C10) were excluded. Light gray dots represent cells excluded from the analysis.

FIG. 12F. Monocle ordered cells on Trajectory 3A, grouped by pseudotime. Both non-basal PCa and CRPC cells were analyzed. Cells from SCNC clusters (C8) and mCRPC cluster (C3) were excluded. Light gray dots represent cells excluded from the analysis FIG. 12G. Monocle ordered cells on Trajectory 3B, grouped by pseudotime. Only non-basal CRPC cells were analyzed. Cells from SCNC clusters (C8) and mCRPC cluster (C3) were excluded. Light gray dots represent cells excluded from the analysis. FIG. 13A-FIG. 13C present data showing the characterization of the evolutionary trajectories of CRPC progression in accordance with one embodiment of the present disclosure.

FIG. 13A. Heatmap of 424 cluster specific markers that were associated with trajectories of CRPC progression. Cells are ordered by pseudotime, shaded by expression level.

FIG. 13B. MSigDB hallmark and oncogenic gene sets enrichment analyses of trajectory associated genes.

FIG. 13C. Enrichr ChIP-X enrichment analyses (ChEA) of trajectory associated genes.

FIG. 14A-FIG. 14F present data showing the evolutionary analyses of CRPC progression in accordance with one embodiment of the present disclosure.

FIG. 14A. Genotype variance (both mutation and SNP) defined evolutionary connection between AR-high CRPC and subpopulation of cells in primary PCa. Left panel shows genotype hierarchical clustering of cells isolated from CRPC1 sample (AR-high CRPC). Right panel shows the distribution of cells with singleton genotype within each of the non-basal clusters.

FIG. 14B. Genotype variance (both mutation and SNP) defined evolutionary connection between PSA-high mCRPC and subpopulation of cells in primary PCa. Left panel shows genotype hierarchical clustering of cells isolated from CRPC3 sample (PSA-high mCRPC). Right panel shows the distribution of cells with singleton genotype within each of the non-basal clusters.

FIG. 14C. CNV defined evolutionary connection between SCNC and subpopulation of cells in primary PCa. Left panel shows CNV hierarchical clustering of cells isolated from CRPC2 sample (SCNC). Right panel shows the distribution of cells with CNV probability >0.65 within each of the non-basal clusters.

FIG. 14D. A model of evolutionary connection between CRPC and subpopulation of cells in primary PCa.

FIG. 14E. Heatmap of average expression 51 evolution signature gene in each cell cluster.

FIG. 14F. Average CRPCsig51 signature score among non-basal cell clusters.

FIG. 15A-FIG. 15F present data showing the characterization of NE cells in primary PCa in accordance with one embodiment of the present disclosure.

FIG. 15A. Monocle decomposed basal, NE and luminal cells of primary PCa by progress through differentiation, colored by cell clusters. Dots represent single cells. Solid lines represent distinct cell trajectories defined by single-cell transcriptomes. Shading represents individual cell cluster. Arrow line represents trajectory direction.

FIG. 15B. Monocle decomposed basal, NE and luminal cells of primary PCa by progress through differentiation, shaded by pseudotime.

FIG. 15C. Monocle ordered cancer NE and luminal cells of primary PCa, shaded by cell clusters.

FIG. 15D. Monocle ordered cancer NE and luminal cells of primary PCa, shaded by pseudotime.

FIG. 15E. Comparison of MSigDB hallmark and oncogenic pathway signaling between PCa cancer and benign cells. Pathway signaling was measured using PC1 of each selected gene set, and differences in pathway signaling between cancer and benign cells was determined using Mann-Whitney U Test (MWU). Direct represents pathway signaling was up-regulated in cancer cells, compared with benign cell. Inverse represents down-regulation.

FIG. 15F. Comparison of MSigDB hallmark and oncogenic pathway signaling between NE cell clusters (C12, C13, C15 and C16) and SCNC cluster (C8).

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element. “About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition (e.g., prostate cancer) manifested by a patient or to which a patient may be susceptible. The aim of treatment includes the alleviation or prevention of symptoms, slowing or stopping the progression or worsening of a disease, disorder, or condition and/or the remission of the disease, disorder or condition (e.g. prostate cancer). As used herein, the terms “prevent,” “preventing,” “prevention,” “prophylactic treatment” and the like refer to reducing the probability of developing a disease, disorder or condition (e.g. prostate cancer) in a subject, who does not have, but is at risk of or susceptible to developing a disease, disorder or condition (e.g., prostate cancer).

The term “effective amount” or “therapeutically effective amount” refers to an amount sufficient to effect beneficial or desirable biological and/or clinical results.

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

As used herein, the term “biomarker” or “biomarkers” refers to a naturally occurring biological molecule(s) present in a subject at varying concentrations useful in predicting the risk or incidence of a disease or a condition. For example, the biomarker(s) can be a gene (or gene signature) that is expressed in higher or lower amounts in a subject at risk for aggressive prostate cancer. The biomarker(s) can include genes, nucleic acids, ribonucleic acids, or a polypeptide used as an indicator or marker for aggressive prostate cancer in the subject.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. A biological sample may be obtained directly from a subject (e.g., by blood or tissue sampling) or from a third party (e.g., received from an intermediary, such as a healthcare provider or lab technician). In some embodiments, the biological sample is a sample of or containing prostate cells, such as a prostate biopsy.

The term “disease” as used herein includes, but is not limited to, any abnormal condition and/or disorder of a structure or a function that affects a part of an organism. It may be caused by an external factor, such as an infectious disease, or by internal dysfunctions, such as cancer, cancer metastasis, and the like. In some embodiments, the disease is a cancer such as prostate cancer.

As is known in the art, a cancer is generally considered as uncontrolled cell growth. The methods of the present invention can be used to treat any cancer, and any metastases thereof, including, but not limited to, carcinoma, lymphoma, blastoma, sarcoma, and leukemia. More particular examples of such cancers include breast cancer, prostate cancer (including castration-resistant prostate cancer [CRPC]), colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma and peripheral neuroepithelioma.

In some embodiments, the cancer comprises prostate cancer. As used herein, the term “aggressive prostate cancer” refers to those types of prostate cancers that are associated with poor event-free survival and/or biochemical recurrence-free survival. Such types of prostate cancer include, but are not limited to, high grade primary PCa, metastatic primary PCa, metastatic CRPC, and the like.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient). In some embodiments, the subject is a human. In certain embodiments, the subject is a human suffering from, or at risk of suffering from, prostate cancer.

As a non-limiting example, the subject may have an early-stage prostate cancer diagnosis (e.g., Stage I or Stage II, and/or cancer has not spread beyond the prostate) based on biopsy results, which is determined to be of intermediate risk. The methods taught herein may aid in determining an appropriate course of treatment for such a subject. Intermediate prostate cancer risk may be defined, for example, as having no features of high-risk prostate cancer and at least one of the following: i) cancer can be felt on a digital rectal exam, but it is only in the prostate and has not spread to your lymph nodes or other organs; ii) Gleason grade group is 3 or lower; and iii) PSA level is between 10 and 20.

Unless otherwise defined, all technical terms used herein have the same meaning as commonly understood by one of ordinary skill in the art to which this disclosure belongs.

The inventors have identified a unique genetic signature that provides for identifying subjects at risk of developing, or who may be already suffering from, an aggressive form of prostate cancer. Based on this information, determination of which treatments, and when to administer such treatment, can be determined thereby better treating the subject.

Accordingly, one aspect of the present disclosure provides a genetic signature for the determination of a disease state in a subject suffering from, or at risk of suffering from, prostate cancer, the genetic signature comprising, consisting of, or consisting essentially of one or more genes provided in Table 1. In one embodiment, the genetic signature comprises one or more genes listed as CRPCsig51_old as provided in Table 1. In another embodiment, the genetic signature comprises one or more genes listed as CRPCsig51_new as provided in Table 1. In some embodiments, the signature comprises one or more of the genes that are in both of CRPCsig51_old and CRPCsig51_new as provided in Table 1.

For example, the signature may comprise from 3, 5, 8 or 10 to 20, 25, 30, 35, 40, 45, or 51 of the genes listed in Table 1 (e.g., from 3 to 51, from 3 to 45, from 3 to 40, from 3 to 35, from 3 to 30, from 3 to 25, from 3 to 20, from 5 to 51, from 5 to 45, from 5 to 40, from 5 to 35, from 5 to 30, from 5 to 25, from 5 to 20, from 8 to 51, from 8 to 45, from 8 to 40, from 8 to 35, from 8 to 30, from 8 to 25, from 8 to 20, from 10 to 51, from 10 to 45, from 10 to 40, from 10 to 35, from 10 to 30, from 10 to 25, or from 10 to 20).

In some embodiments, the signature comprises from 3, 5, 8 or 10 to 20, 25, 30, 35, 40, 45, or 51 of the genes listed as CRPCsig51_old in Table 1 (e.g., from 3 to 51, from 3 to 45, from 3 to 40, from 3 to 35, from 3 to 30, from 3 to 25, from 3 to 20, from 5 to 51, from 5 to 45, from 5 to 40, from 5 to 35, from 5 to 30, from 5 to 25, from 5 to 20, from 8 to 51, from 8 to 45, from 8 to 40, from 8 to 35, from 8 to 30, from 8 to 25, from 8 to 20, from 10 to 51, from 10 to 45, from 10 to 40, from 10 to 35, from 10 to 30, from 10 to 25, or from 10 to 20).

In some embodiments, the signature comprises from 3, 5, 8 or 10 to 20, 25, 30, 35, 40, 45, or 51 of the genes listed as CRPCsig51_new in Table 1 (e.g., from 3 to 51, from 3 to 45, from 3 to 40, from 3 to 35, from 3 to 30, from 3 to 25, from 3 to 20, from 5 to 51, from 5 to 45, from 5 to 40, from 5 to 35, from 5 to 30, from 5 to 25, from 5 to 20, from 8 to 51, from 8 to 45, from 8 to 40, from 8 to 35, from 8 to 30, from 8 to 25, from 8 to 20, from 10 to 51, from 10 to 45, from 10 to 40, from 10 to 35, from 10 to 30, from 10 to 25, or from 10 to 20).

In some embodiments, the genetic signature comprises from 3, 4, 5, or 6, to 8, 9, 10, 11, or 12 of the genes listed in Table 4 (e.g., from 3 to 12, from 3 to 11, from 3 to 10, from 3 to 9, from 3 to 8, from 4 to 12, from 4 to 11, from 4 to 10, from 4 to 9, from 4 to 8, from 5 to 12, from 5 to 11, from 5 to 10, from 5 to 9, from 5 to 8, from 6 to 12, from 6 to 11, from 6 to 10, from 6 to 9, or from 6 to 8).

The genetic signature can be prepared by obtaining a biological sample from a subject, and, using methods known to those skilled in the art and as detailed herein in the Examples, compiling the genetic signature and measuring expression levels of the biomarkers of the signature. In some embodiments, the resulting measurement is compared to a control, which may be, for example, measurement of the signature from a subject not suffering from, or at risk of suffering from, aggressive prostate cancer, measurement of the signature from non-cancerous prostate tissue of the subject, etc. As a non-limiting example, up-regulated genes in the signature may be determined using the Wilcox test (Bonferroni adjusted P<0.01, and >2-fold higher expression than control).

By comparing the measurement of the genetic signature (e.g., a higher mRNA and/or protein expression level of genes in the signature) obtained from the biological sample of a subject with that of a control sample, a score can be calculated (see, e.g., the Examples, which use Youden's index to dichotomize CRPCsig51 into low and high scores). Subjects having a higher score (which may indicate a larger proportion of the tissue comprising cells having a CRPC evolutionary signature) are suffering from, or at risk of suffering from, aggressive prostate cancer, which may indicate more aggressive treatment options “Score” as used herein may be any measurement output representation of a higher expression level of the one or more genes making up the signature, indicative of a higher risk of disease progression. In some embodiments, the measurement output represents maximum specificity and sensitivity

Hence, another aspect of the present disclosure provides a method of determining the risk of disease progression in a subject suffering from, or at risk of suffering from, prostate cancer, the method comprising, consisting of, or consisting essentially of (i) obtaining a biological sample from the subject; and (ii) determining the CRPC evolutionary signature in the sample to generate a score, in which samples having a high score as compared to a control is indicative of aggressive prostate cancer.

Based on the score obtained, one skilled in the art can determine whether to administer a prostate cancer treatment early and/or whether aggressive prostate cancer treatments should be administered. Thus, another aspect of the present disclosure provides a method of determining the risk of disease progression in a subject suffering from, or at risk of suffering from, prostate cancer, the method comprising, consisting of, or consisting essentially of (i) obtaining a biological sample from the subject; (ii) determining the CRPC evolutionary signature in the sample to generate a score, in which samples having a high score as compared to a control is indicative of aggressive prostate cancer and (iii) administering early and/or aggressive prostate cancer treatments to the subject if the score is higher than that of the control.

Yet another aspect of the present disclosure provides a method of determining whether early and/or aggressive prostate cancer treatments should be administered to a subject, the method comprising, consisting of, or consisting essentially of (i) obtaining a biological sample from the subject; (ii) determining the CRPC evolutionary signature in the sample to generate a score, in which samples having a high score as compared to a control is indicative of aggressive prostate cancer and (iii) administering early and/or aggressive prostate cancer treatments to the subject if the score is higher than that of the control.

In one embodiment, the CRPC evolutionary signature comprises at least one or more genes provided in Table 1, as noted above.

There are numerous approved treatments for prostate cancer. The type of treatment, dosage amounts and/or frequency, and the like are dependent on the particular subject and type/stage of disease being treated and can be readily determined by one skilled in the art without undue experimentation. As used herein, the term “aggressive prostate cancer treatment” refers to those treatments commonly administered by those skilled in the art to subjects suffering from an aggressive prostate cancer. Suitable treatment regimens for prostate cancer and aggressive prostate cancer include, but are not limited to, observation, castration (including orchiectomy [surgical castration] and chemical castration [e.g., LHRH agonists such as Leuprolide, Goserelin, Triptorelin, Leuprtolide mesylate, etc. and LHRH antagonists such as Degarelix, Relugolix, etc.], chemotherapy (e g., using chemotherapeutic agents such as docetazel, cavazitaxel, mitoxantrone, estramustine, etc.), hormone therapy (e.g., androgen deprivation/suppression therapy), immunotherapy (e.g., check-point inhibitors, cancer vaccines such as sipuleucel-T, etc.), radiation, surgery, cryotherapy, targeted therapy (e.g., PARP inhibitors such as Rucaparib, Olaparib, etc.), and the like.

In some embodiments, the prostate cancer treatment, such as for an aggressive prostate cancer, may include one or more of surgery (e.g. prostatectomy such as radical prostatectomy), radiation (e.g., external beam radiation therapy (EBRT) or brachytherapy), and focal treatment (e.g., heat, cold, or laser treatment of the prostate tissue).

Other aspects of the present disclosure provide for assays for determining the genetic signature (e.g., the CRPCsig51 genetic signature) of a subject. Generally, such assays a method, the method comprising, consisting of, or consisting essentially of (i) obtaining a biological sample from the patient; (ii) determining a genetic signature from the biological sample; and (iii) providing an output comprising the genetic signature. Assays as provided herein can be achieved using a number of known techniques, including, but not limited to, PCR, northern blotting, southern blotting, and the like.

The present disclosure further provides kits for carrying out the subject methods as provided herein. For example, in one embodiment, a subject kit may comprise, consist of, or consist essentially of, for example, (i) primers specific for one or more of the CRPCsig51 genes; (ii) antibodies specific for one or more of the CRPCsig51 gene products, and the like.

In other embodiments, a kit may further include other components. Such components may be provided individually or in combinations, and may provide in any suitable container such as a vial, a bottle, or a tube. Examples of such components include, but are not limited to, (i) one or more additional reagents, such as one or more dilution buffers; one or more reconstitution solutions; one or more wash buffers; one or more storage buffers, one or more control reagents and the like, (ii) one or more control samples, such as RNA polynucleotides, DNA nucleotides, etc.; (iii) one or more reagents for in vitro production and/or maintenance of the of the molecules, cells etc. provided herein; and the like. Components (e.g., reagents) may also be provided in a form that is usable in a particular assay, or in a form that requires addition of one or more other components before use (e.g. in concentrate or lyophilized form). Suitable buffers include, but are not limited to, phosphate buffered saline, sodium carbonate buffer, sodium bicarbonate buffer, borate buffer, Tris buffer, MOPS buffer, HEPES buffer, and combinations thereof.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e, associated with the packaging or subpackaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Another aspect of the present disclosure provides all that is described and illustrated herein.

The following Examples are provided by way of illustration and not by way of limitation.

### EXAMPLES

### 1. Cellular Heterogeneity of Prostate Cancer Contributes to Therapy Resistance and Disease Progression

### A. Abstract

Background. Hormonal therapy targeting the androgen receptor (AR) inhibits prostate cancer (PCa) but the tumor eventually recurs as castration-resistant prostate cancer (CRPC).

Objective. To understand the mechanisms by which sub-clones within early PCa develop into CRPC.

Design, setting, and participants. We isolated epithelial cells from fresh human PCa cases, including primary adenocarcinoma, locally recurrent CRPC, and metastatic CRPC and utilized single cell RNA sequencing to identify subpopulations destined to become either CRPC-adeno or small cell neuroendocrine carcinoma (SCNC).

Outcome measurements and statistical analysis. We revealed dynamic transcriptional reprogramming that promotes disease progression among 23,226 epithelial cells using single-cell RNA sequencing, and validated subset-specific progression using immunohistochemistry and large cohorts of publically available genomic data. Results and limitations. We identified a small fraction of highly plastic CRPC-like cells in hormone-naïve early PCa and demonstrated its correlation with biochemical recurrence and distant metastasis, independent of clinical characteristics. We show that progression towards castration-resistance was initiated from subtype specific lineage plasticity and clonal expansion of pre-existing neuroendocrine (NE) and CRPC-like cells in early PCa.

Conclusions. CRPC-like cells are present early in the development of prostate cancer and are not exclusively the result of acquired evolutionary selection during androgen deprivation therapy. The lethal CRPC and SCNC phenotypes should be targeted earlier in the disease course of patients with prostate cancer.

Patient summary. Here we report the presence of pre-existing CRPC-like cells in primary PCa, which represents a novel castration-resistant mechanism different from the adaptation mechanism post ADT. Patients whose tumors harbor increased pre-existing NE and CRPC-like cells may become rapidly resistant to ADT, and may require aggressive early intervention.

### B. Results

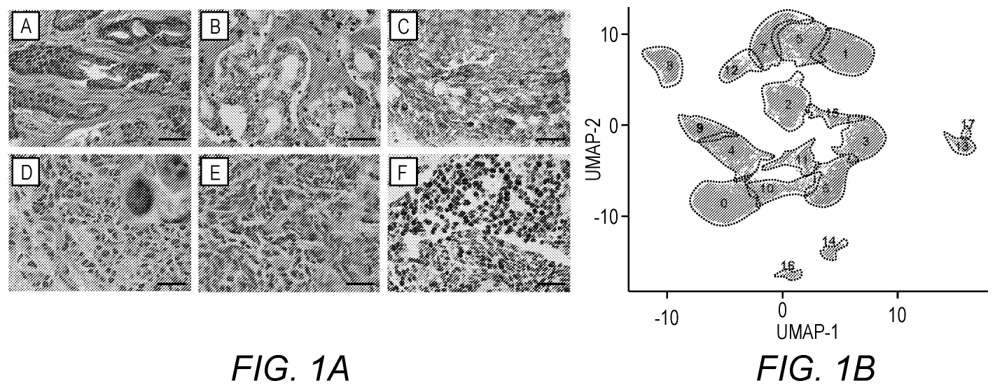

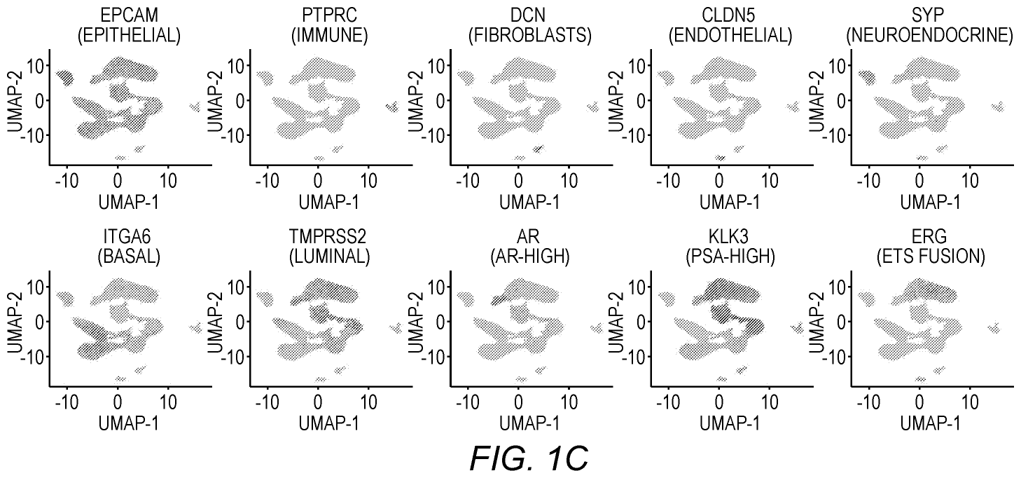

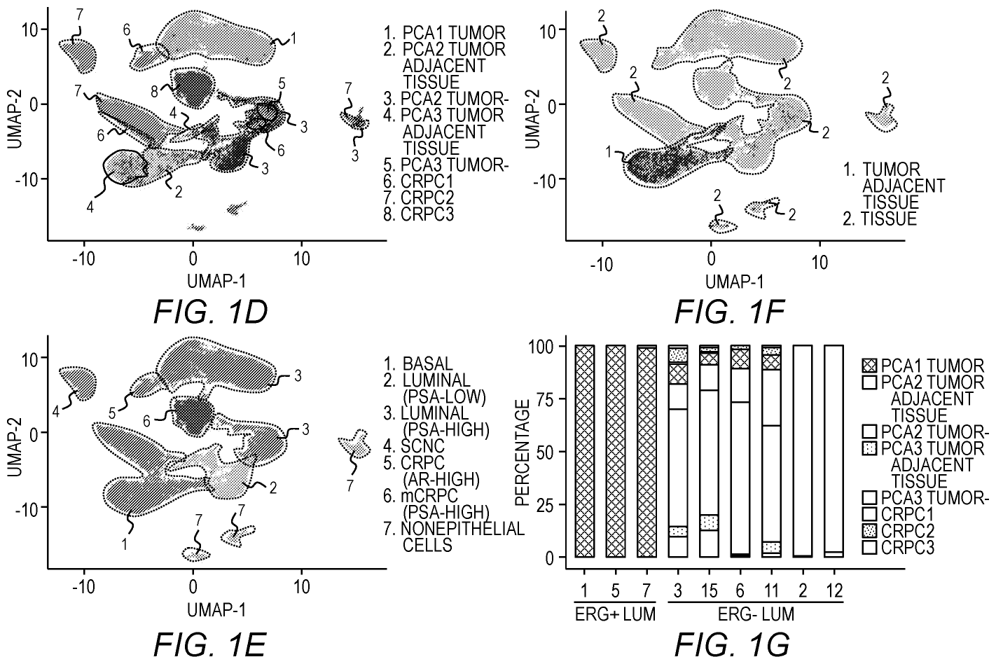

Intra-tumor heterogeneity in primary PCa and CRPC: To characterize intra-tumor heterogeneity as it relates to therapy resistance and disease progression, we collected fresh tissue from the entire spectrum of PCa, including three cases of hormone-naïve primary adenocarcinoma (Gleason Score 4+3); two cases of post-hormonal therapy locally recurrent CRPC with CRPC-adeno and SCNC histology, respectively; and one metastatic CRPC (mCRPC-adeno, to the soft tissue of pelvic sidewall) (FIG. 1A). For the SCNC case, the patient's original tumor was diagnosed as prostatic adenocarcinoma and treated with hormonal therapy, but the recurrent tumor showed classic SCNC histology.

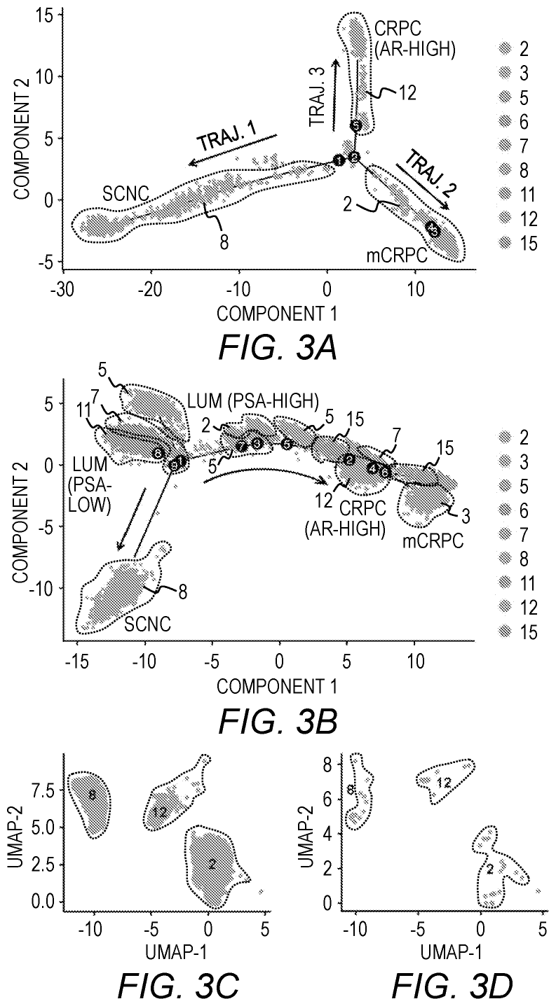

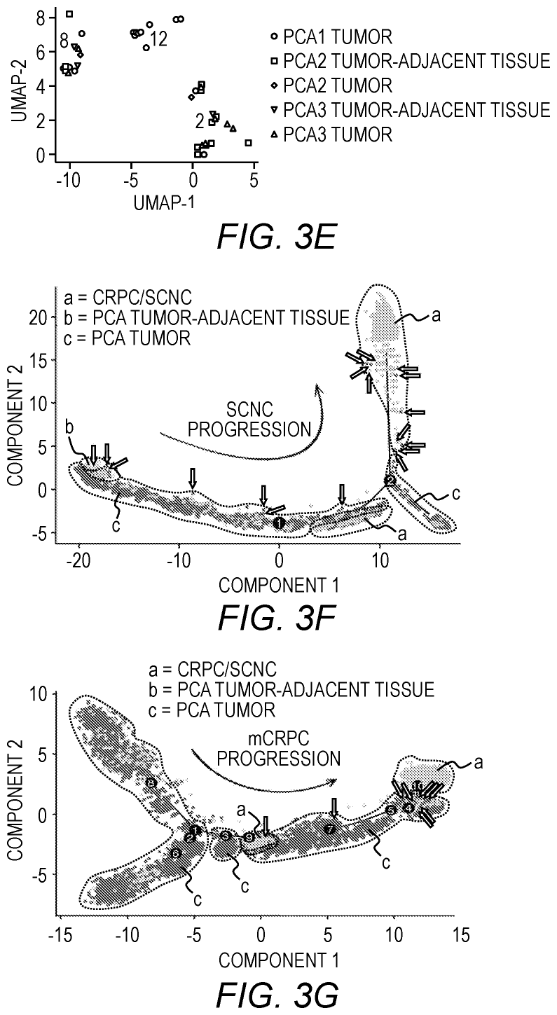

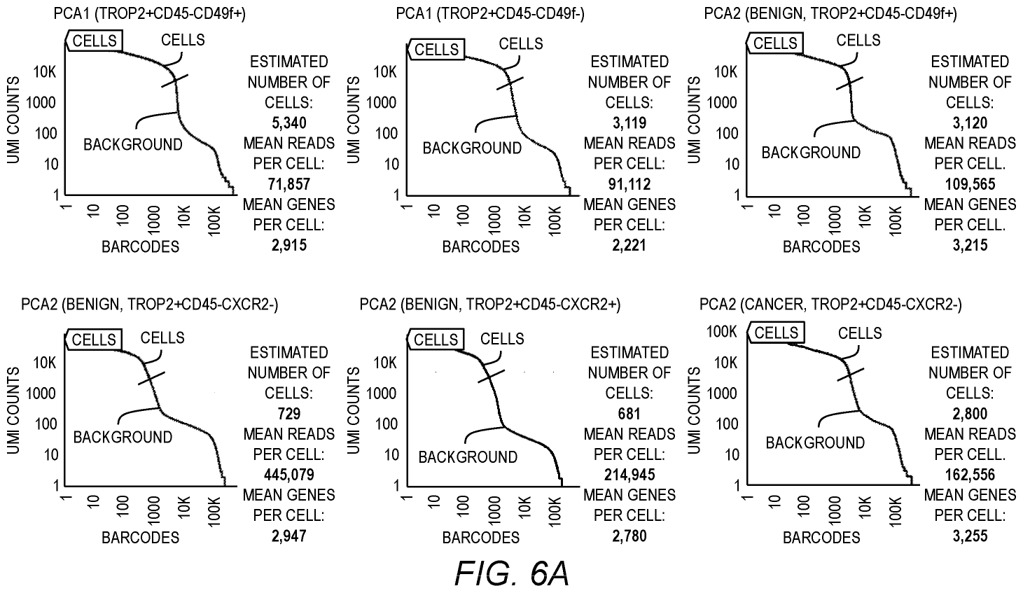

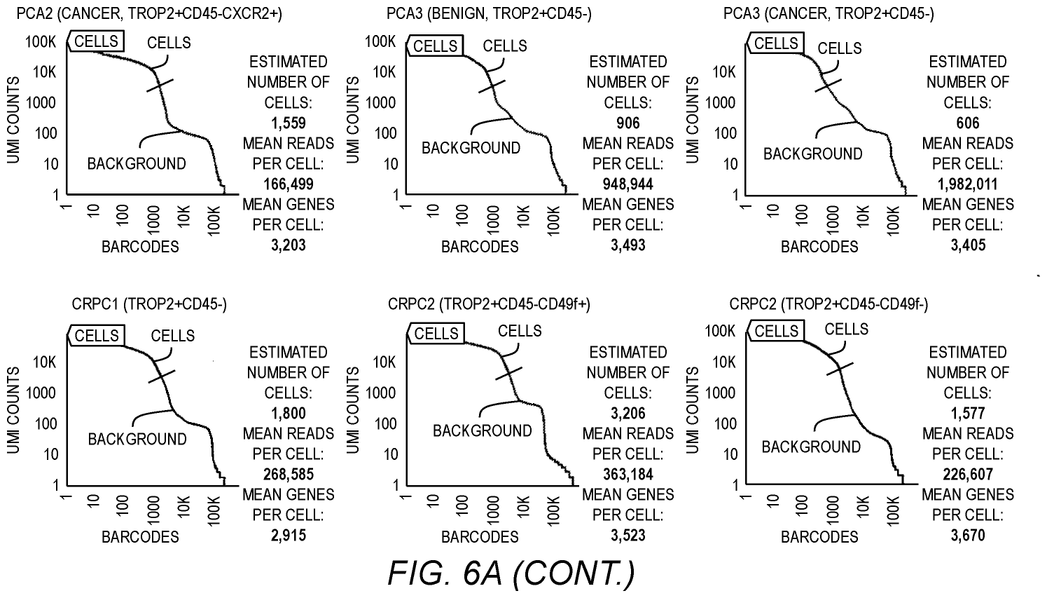

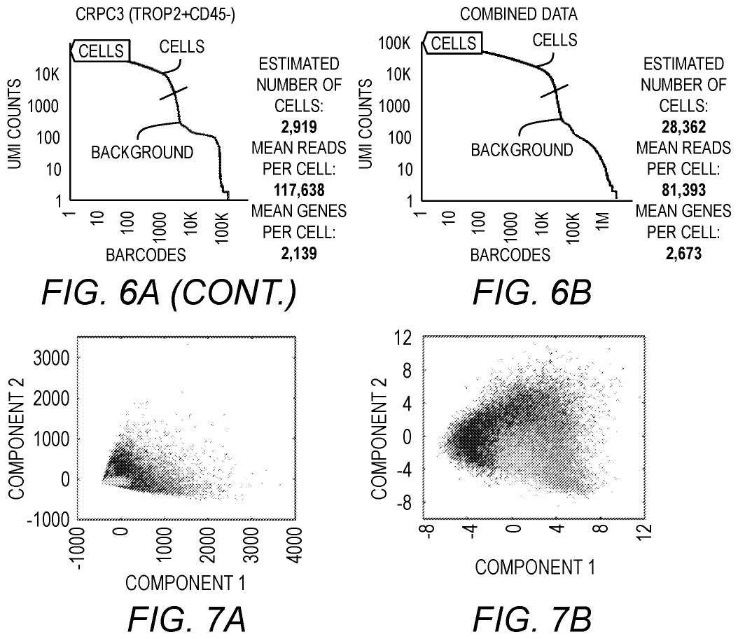

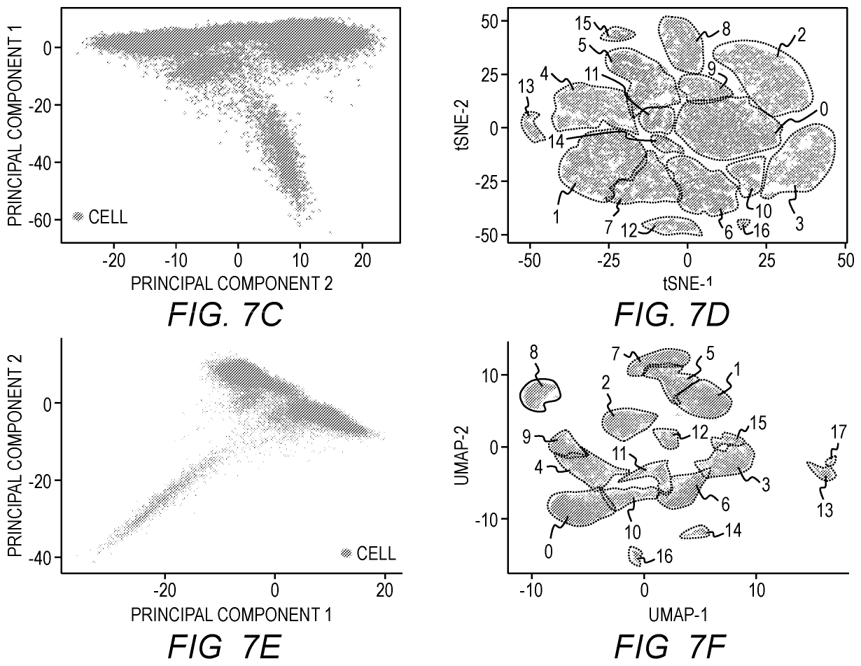

We isolated multiple epithelial cells (Trop2+CD45−) from each sample by FACS and performed droplet-based scRNA-seq (FIG. 6A, 6B). After removing cells with minimum and maximum thresholds for unique molecular identifier (UMI), nGene, and mitochondrial RNA genes, we obtained 24,385 single cells, and no significant batch effects were observed (FIG. 7A-FIG. 7F). Using the Seurat feature of integrating data analyses from different conditions, we identified 18 distinct cell clusters (FIG. 1B) that correspond to subset cluster specific markers (FIG. 3A).

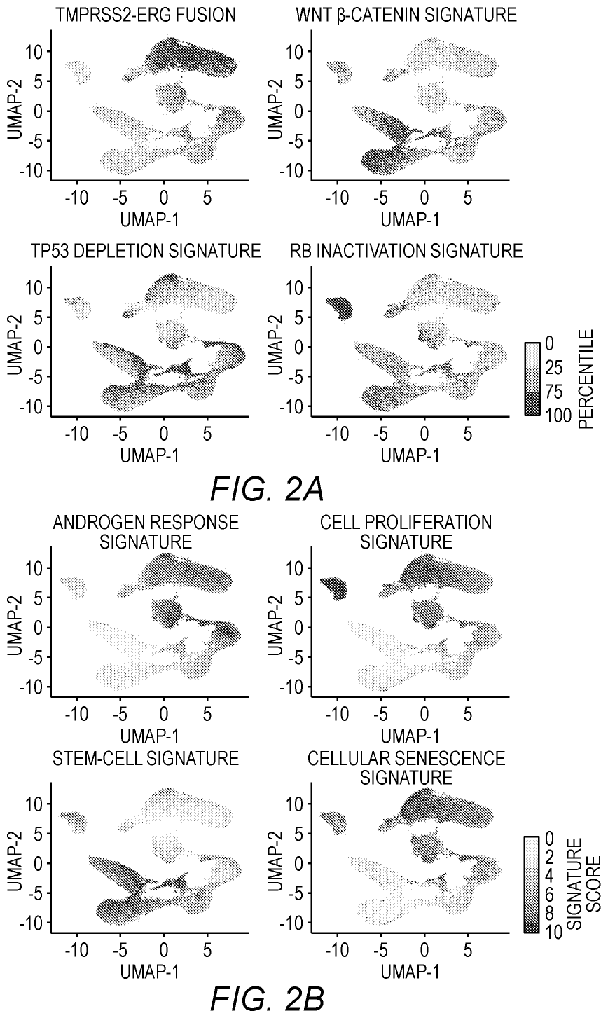

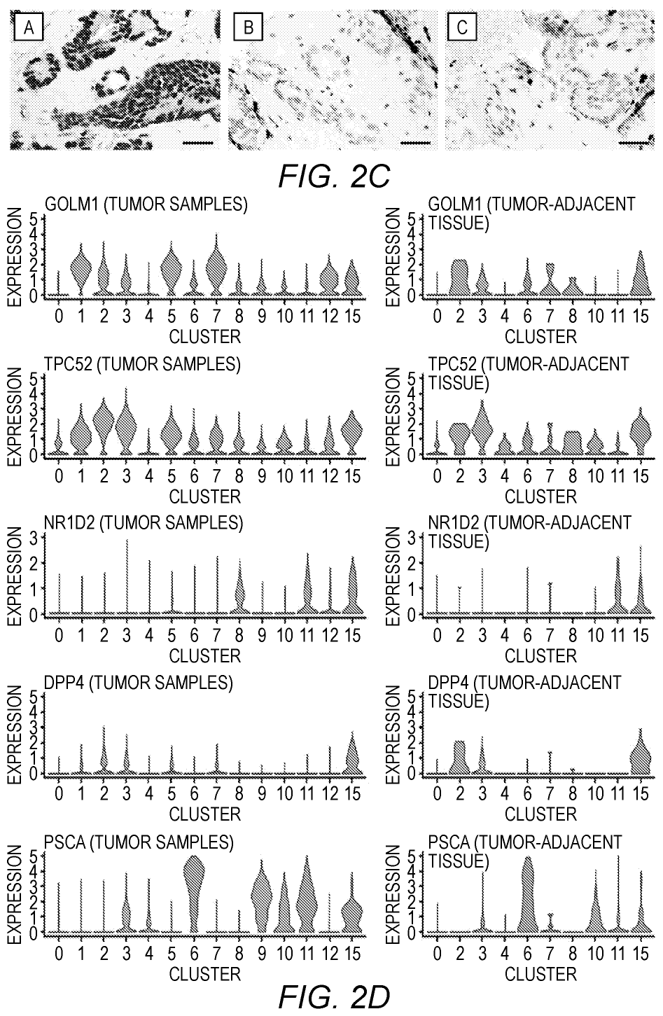

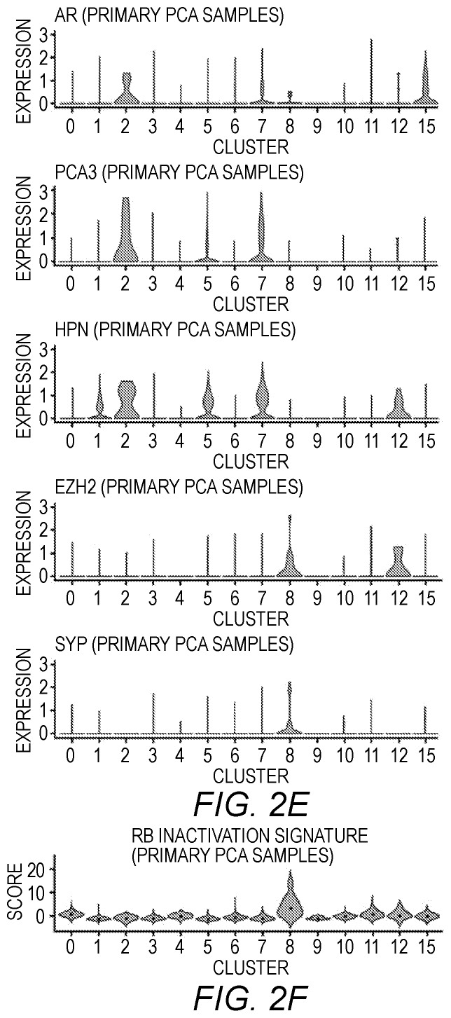

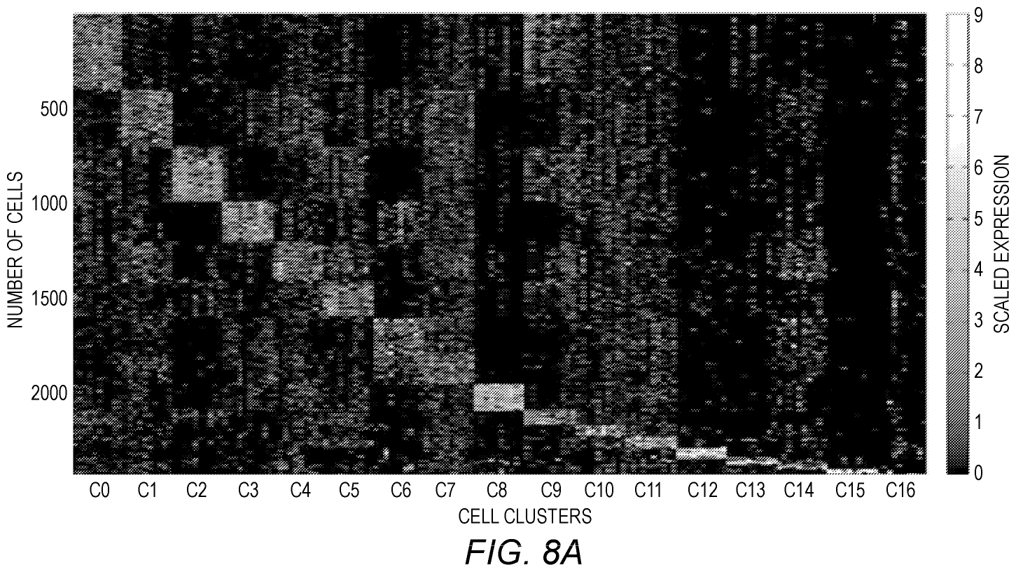

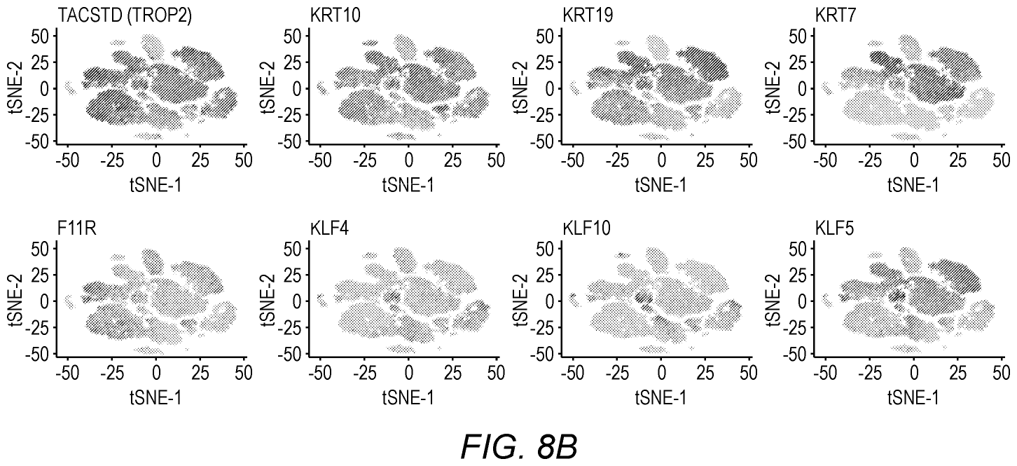

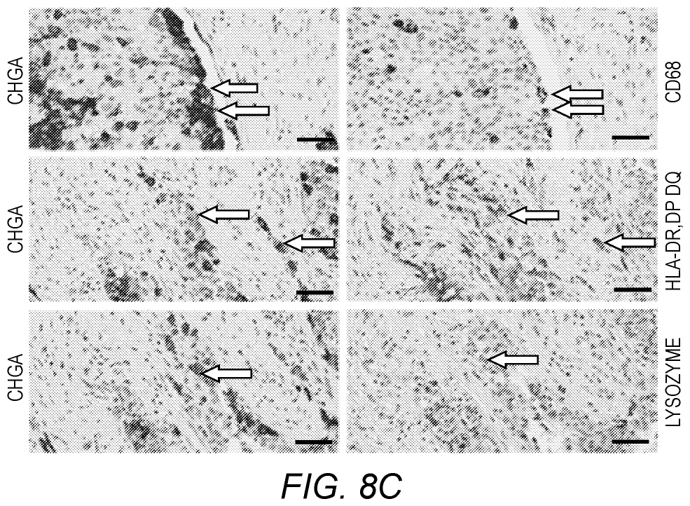

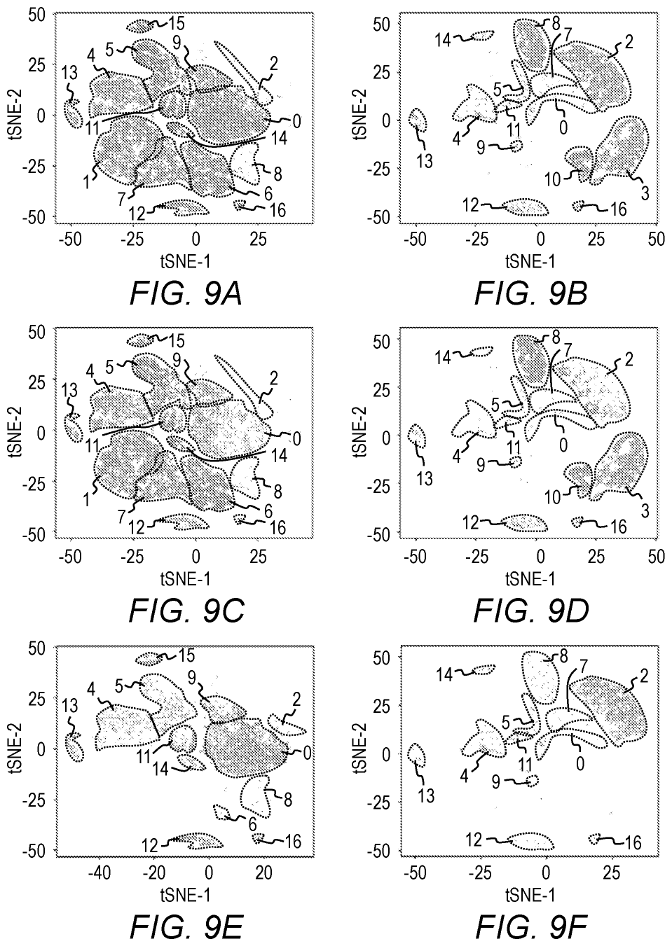

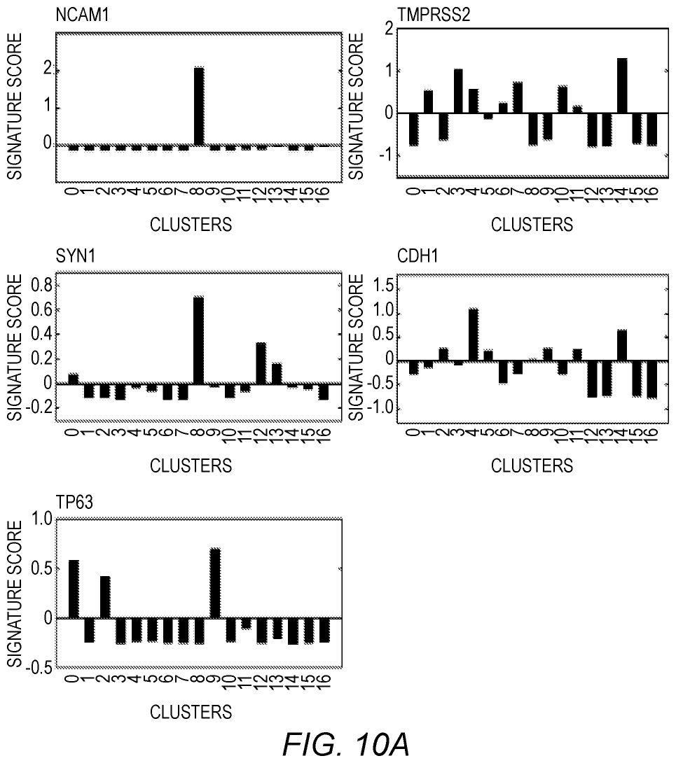

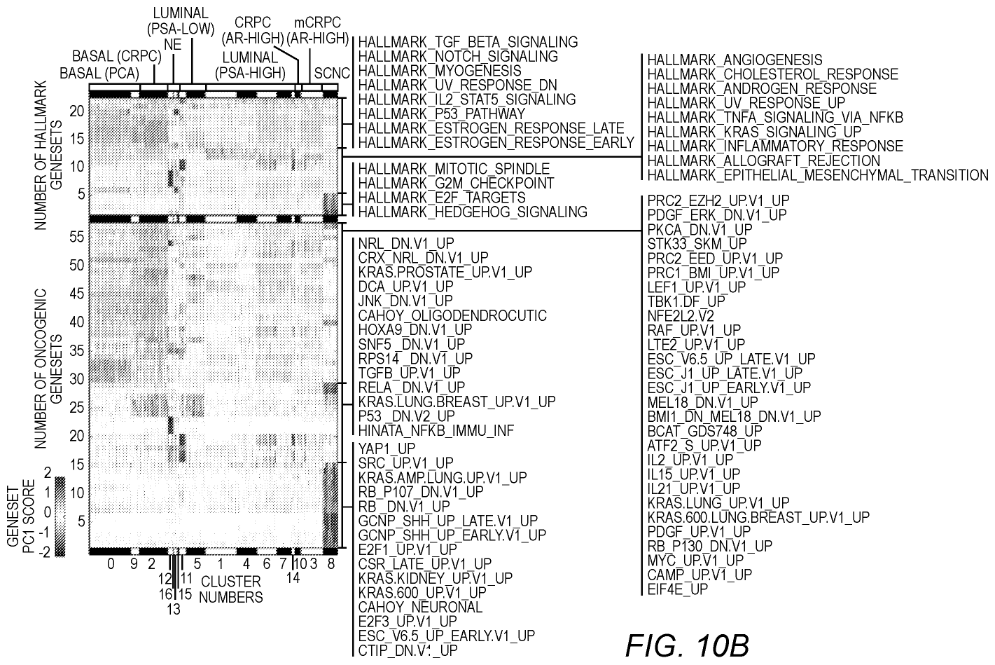

After removing clusters of leukocytes (C13 and C17), fibroblasts (C14), and endothelial cells (C16) (FIG. 1C, FIG. 9A-FIG. 9F), we defined cellular lineage classes of each cluster using the expression of lineage markers and the origin of the cells (FIG. 1C-FIG. 1D, FIG. 10A). We identified multiple subpopulations of PSA-high luminal (C1, C3, C5, C7 and C15), PSA-low luminal (C6, and C11) and basal cells (C0 and C10) from primary PCa, as well as mCRPC (C2), locally recurrent CRPC-adeno (AR-high, C12) or SCNC (C8), and basal cells from CRPC (C4 and C9) (FIG. 1F) Interestingly, we found activation of multiple oncogenic signals in basal cells isolated from CRPC samples, indicating the potential for lineage plasticity in these cells (Table 5). We observed 3 PSA-high luminal clusters (C1, C5 and C7) that formed a separate cell population from other luminal clusters, and over 99% of cells in these clusters were from the PCal sample (FIG. 1D). Analyzing the status of gene fusion of ETS-family genes, we found that up-regulated ERG expression in C1, C5 and C7 was associated with the positive TMPRSS2-ERG fusion signature (FIG. 1C, FIG. 2A, FIG. 8B, FIG. 10B). We confirmed that ERG was overexpressed using IHC analysis (FIG. 2C). We thus classified C1, C5, and C7 as ERG+ luminal clusters, and other luminal clusters as ERG-subtypes.

Although the majority of the cells from tumor-adjacent tissue obtained from ERG-PCa2 and PCa3 cases were basal cells, 23% of these cells were luminal cells (FIG. 1F). Instead of forming distinct non-malignant luminal clusters, the luminal cells from tumor-adjacent tissue were mixed into each of the tumor cell clusters (FIG. 1G), suggesting that ERG-primary PCa shared similar patterns of cellular heterogeneity in tumor and adjacent tissue, while ERG+ luminal clusters tend to be patient-specific.

Tumor cell heterogeneity is related to therapy resistance and disease progression: To identify potential functional differences among the cell clusters, we evaluated a wide series of RNA expression signatures using MSigDB that are up-regulated in cancer including stem cell (n=45), cellular proliferation (n=36), and senescence (n=7) (Table 6). We observed up-regulated androgen response and proliferation signatures in PSA-high luminal clusters, compared with basal and PSA-low luminal clusters (FIG. 2B, FIG. 8B). The higher proliferation in PSA-high luminal clusters was correlated with activated expression of TPD52 and GOLM1, oncogenes that promote PCa proliferation (FIG. 2D). The highest proliferation signatures were seen in cells from SCNC cluster (C8), consistent with the previous finding that SCNC cells are highly proliferative and extremely aggressive. However, the PSA-low clusters (C6 and C11) are unlikely to be aged cells, as the lower proliferation was not correlated with up-regulated senescence signatures (FIG. 2B, FIG. 8B). Cells in cluster C11 possessed stem-cell features, and activated Wnt/β-catenin and EMT signaling (FIG. 2A, FIG. 2B, FIG. 8B). The TP53 deficient signaling and up-regulated NR1D2 expression in C11 and C15 (FIG. 2A, FIG. 2D) suggested lineage plasticity in these two clusters.

A recent study of murine PCa identified two luminal populations (Dpp4+/Prom1+ and Psca+/Krt4+) that contributed to androgen-induced regeneration post castration. A subtype of Psca+ luminal cells was also defined as a progenitor of PCa. We found that the expressions of both DPP4 and PSCA were highly enriched in C15, and up-regulated PSCA in other luminal clusters (C3, C6 and C11) was progressively increased from tumor-adjacent tissue to PCa, indicating that each of these luminal cell subtypes may participate in tumorigenesis (FIG. 2D).

The critical signaling network of disease progression towards either CRPC-adeno or SONC: We next studied potential correlations between intrinsic cellular heterogeneity and disease progression to CRPC/SCNC. Using the Monocle reverse graph embedding approach, non-basal CRPC/SCNC cells were decomposed into a trifurcated architecture of the cell trajectory, moving toward SCNC (C8), locally recurrent CRPC-adeno with AR-high (C12), and mCRPC with PSA-high clusters (C2) (FIG. 3A). When both non-basal PCa and CRPC cells were integrated into the evolutionary trajectory analysis, a bifurcated architecture of AR-dependent and AR-independent cell trajectory was observed, in which locally recurrent CRPC-adeno cells were located at the interphase on the branch progressing toward mCRPC cluster (FIG. 3B).

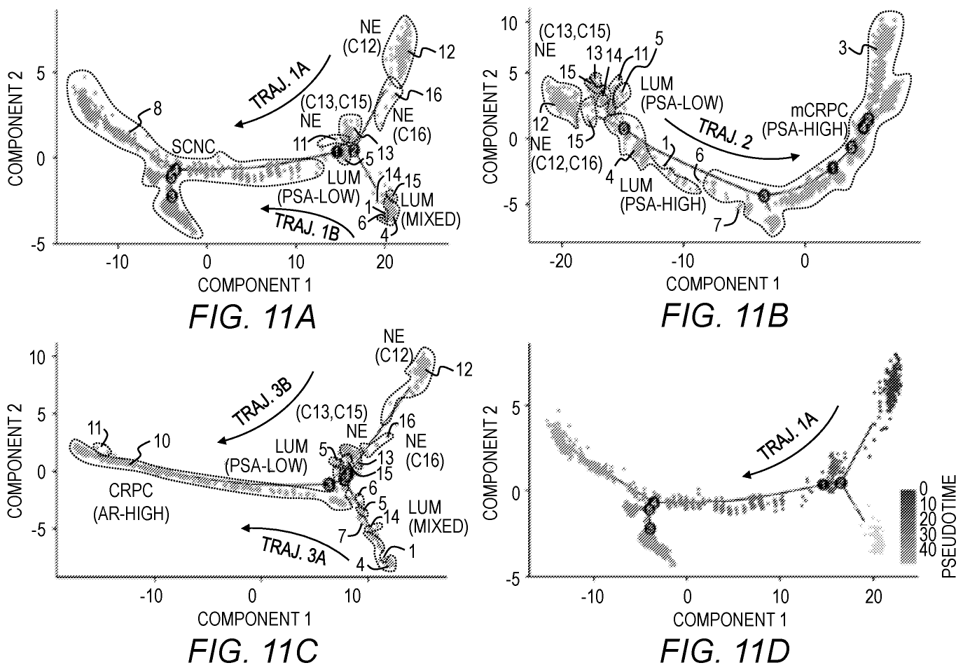

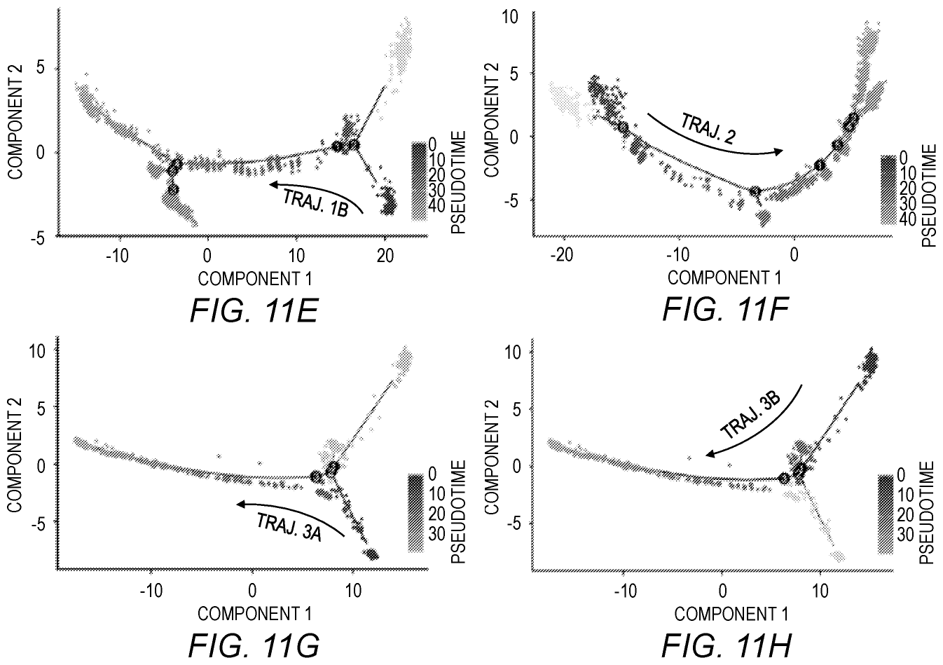

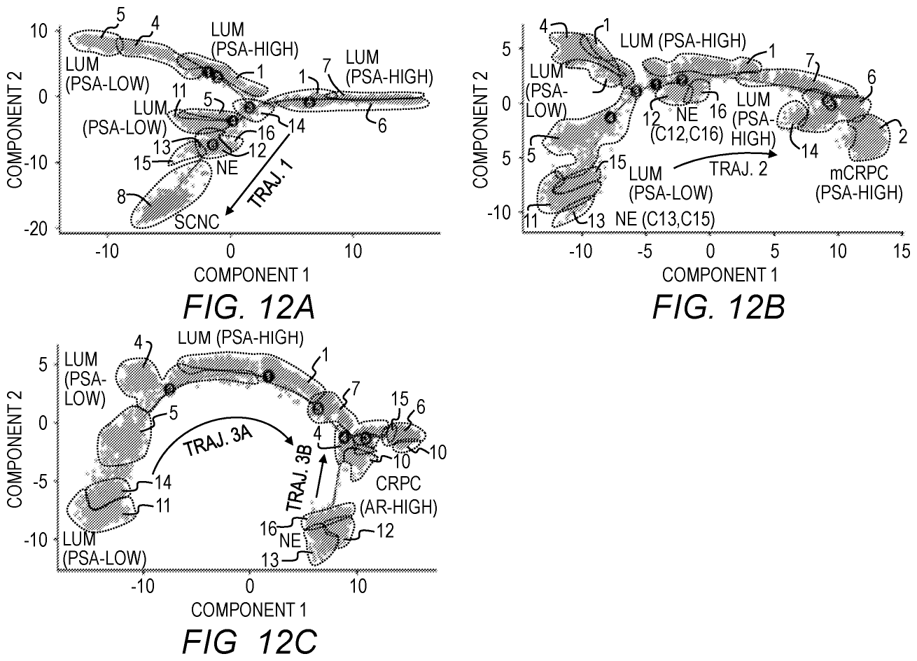

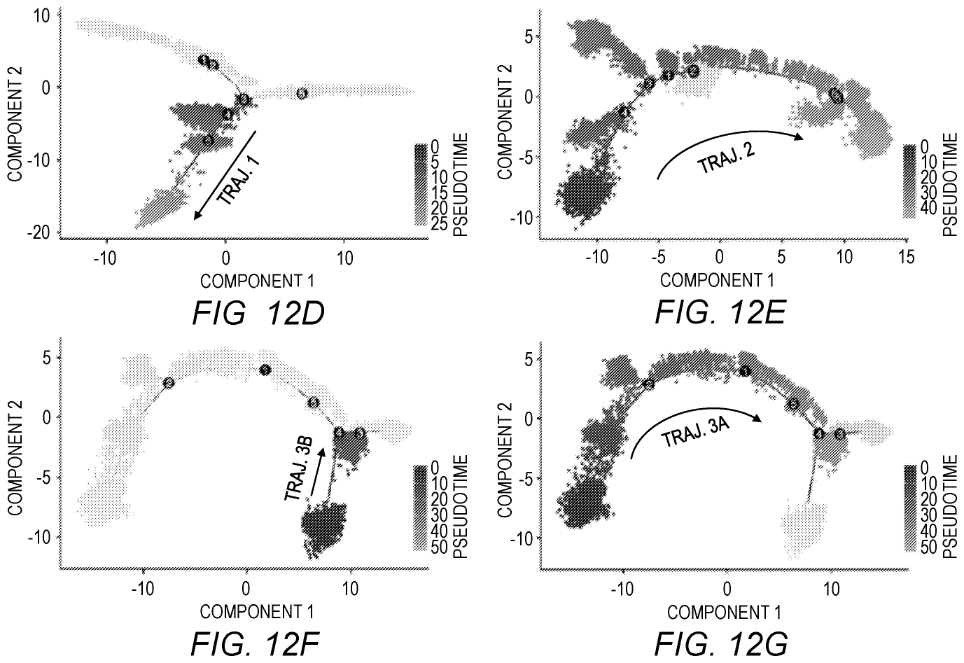

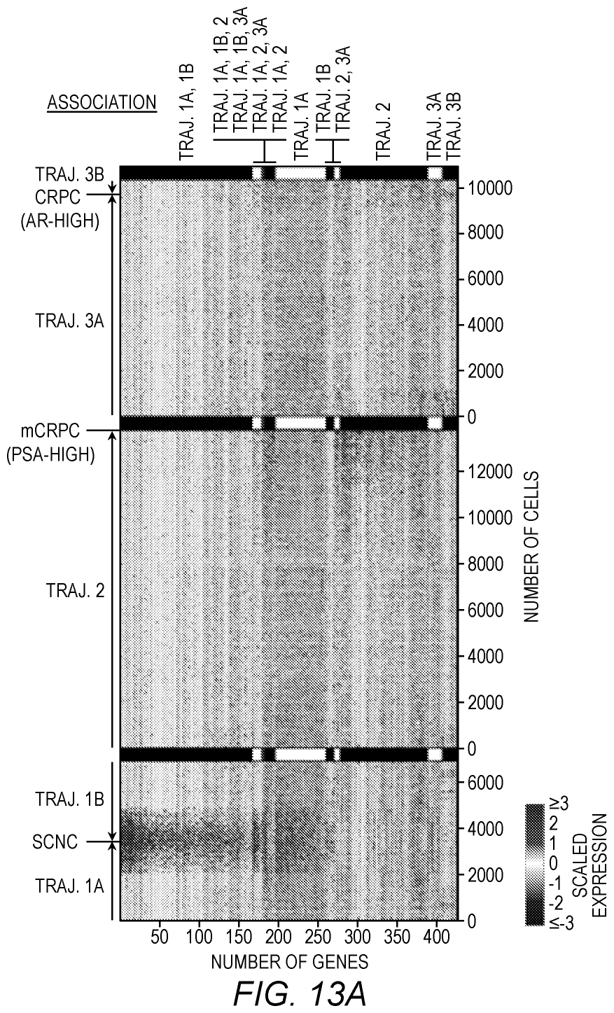

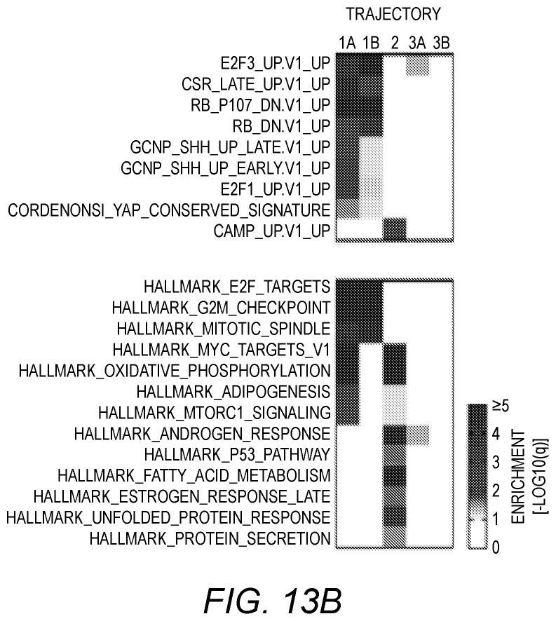

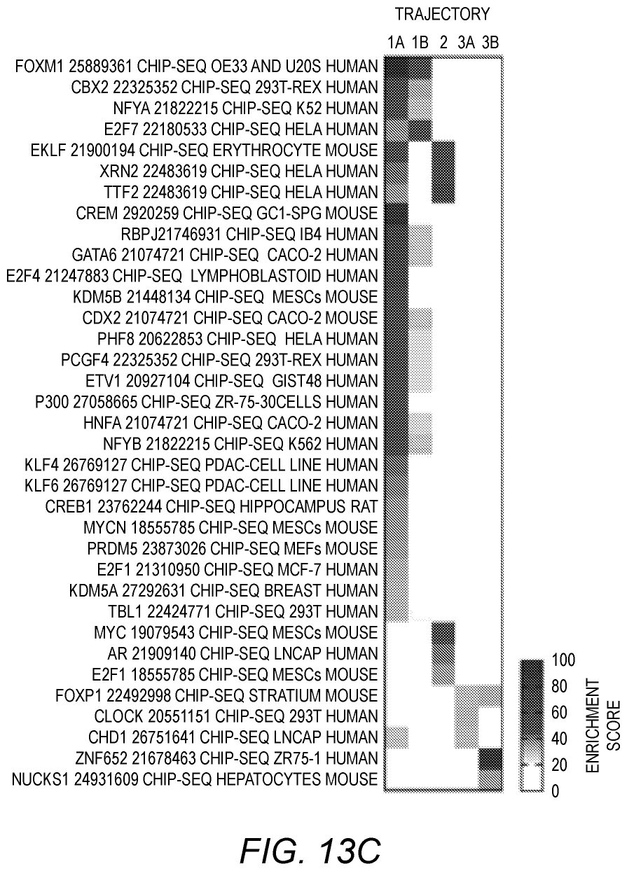

To validate evolutionary trajectories, we performed ROC analyses to determine if any of the clusters carries the CRPC/SCNC signature (Table 7, and FIG. 11A). Hierarchical relationships among cell clusters were further assessed using genotype (SNP-mutation) and CNV patterns of the same CRPC/SCNC patients. (FIG. 11B-FIG. 11E). We found that a multipotent stem-like PSA-low luminal cluster (C11) gave rise to both treatment-emergent SCNC and CRPC-adeno, while progression of CRPC-adeno could also originate from multiple subpopulations including PSA-high luminal (C3 and C15) and PSA-low luminal cell (C6) clusters FIG. 11F). By comparing the changes in relative gene expression over the five major dynamic-processes (FIG. 12A-FIG. 12G, FIG. 13A-FIG. 13C), we revealed 1,705 genes whose expression was associated with one or more trajectories, and identified 13 critical transcription factors (TFs) that modulated progression to SCNC/CRPC (FIG. 11G).

The presence of rare CRPC-like cells in primary PCa: By analyzing up to 7949 epithelial cells per patient sample (FIG. 8C), we observed that a small number of primary PCa cells (52 cells) clustered into CRPC/SCNC clusters (C2, C8 and C12) (FIG. 3C-FIG. 3E). Using the expression of well-established PCa markers, we confirmed that these were not misclassified noise cells (FIG. 2E). Based on positive expression of SYP and EZH2, and inactivated RB1 signaling, we classified the primary PCa cells in the C8 cluster as NE cells (FIG. 2E-FIG. 2F). The primary PCa cells with up-regulated expression of AR, HPN and PCA3 in CRPC-adeno clusters (C2 and C12) were defined as CRPC-like cells (FIG. 2E). We found that NE cells from primary PCa were distributed on each state of NED trajectory toward SCNC (FIG. 3F), suggesting that the NE cells have the ability of self-renewal that can accelerate clonal expansion under the pressure of hormonal therapy. Unlike NE cells, 16 of 19 (84%) CRPC-like cells in cluster C2 resided next to the end state of mCRPC trajectory, indicating that these cells are fully progressed CRPC cells in primary PCa that will promote resistance to hormonal therapy (FIG. 3G).

### CRPCsig51 Signature

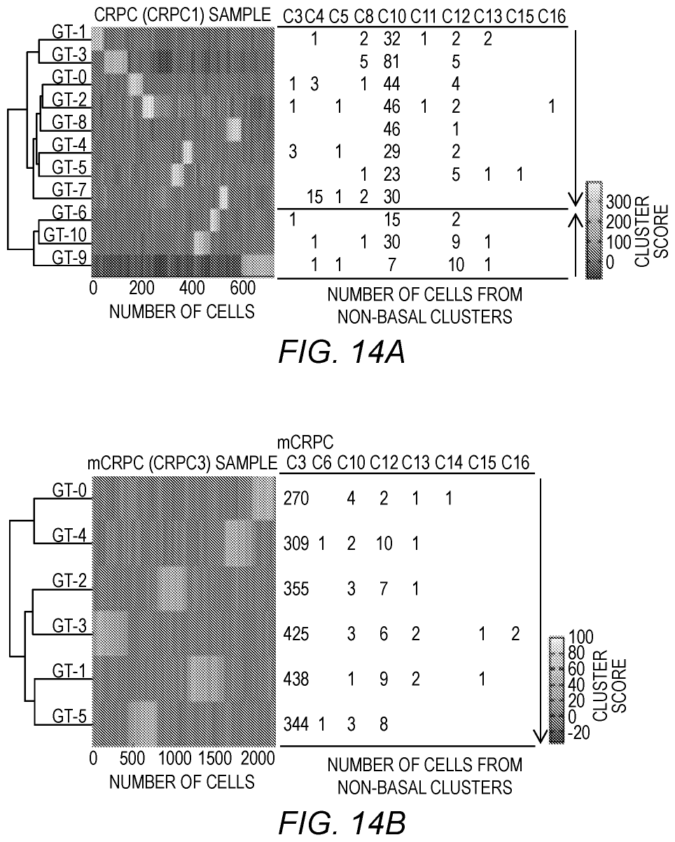

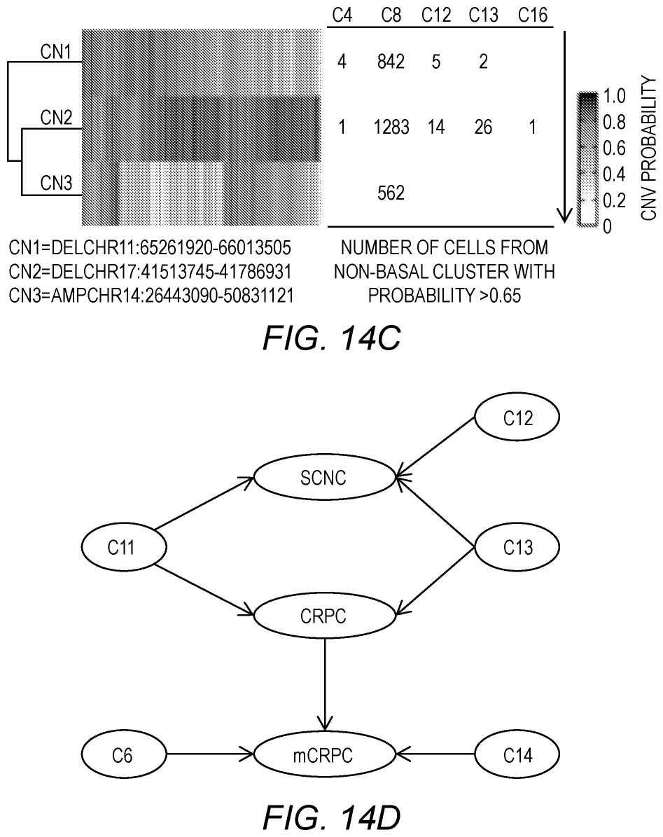

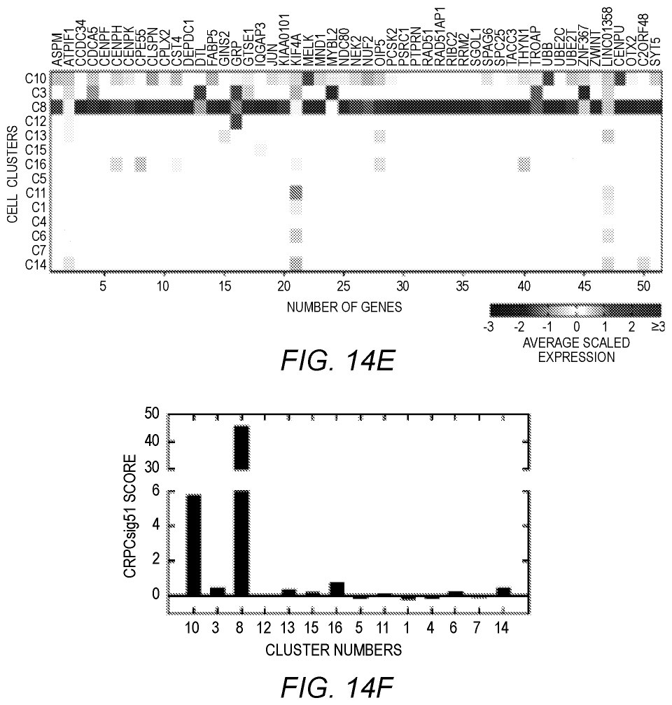

To further determine the correlation between pre-exiting CRPC-like cells and castration resistance, we developed a novel signature called CRPCsig51 using 51 genes that were significantly up-regulated in CRPC-like cells and associated with the CRPC/SCNC evolutionary trajectory (FIG. 14A-FIG. 14B). Cells were clustered using tSNE (Table 1: CRPCsig51_old), and data were re-analyzed using a newer version of Seurat that defines clusters using UMAP (Table 1: CRPCsig51_new). Validation proceeded with the CRPCsig51_new set of genes.

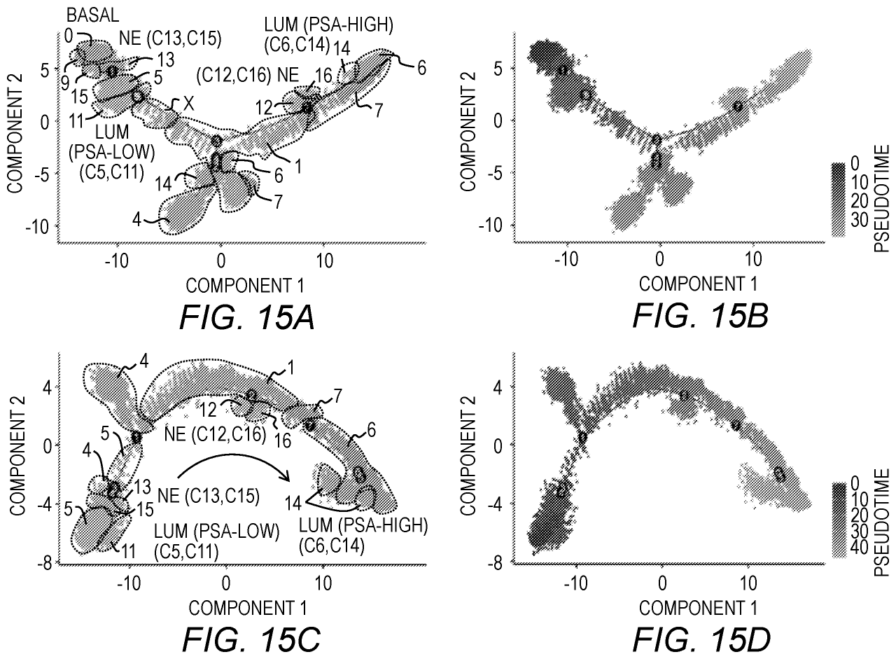

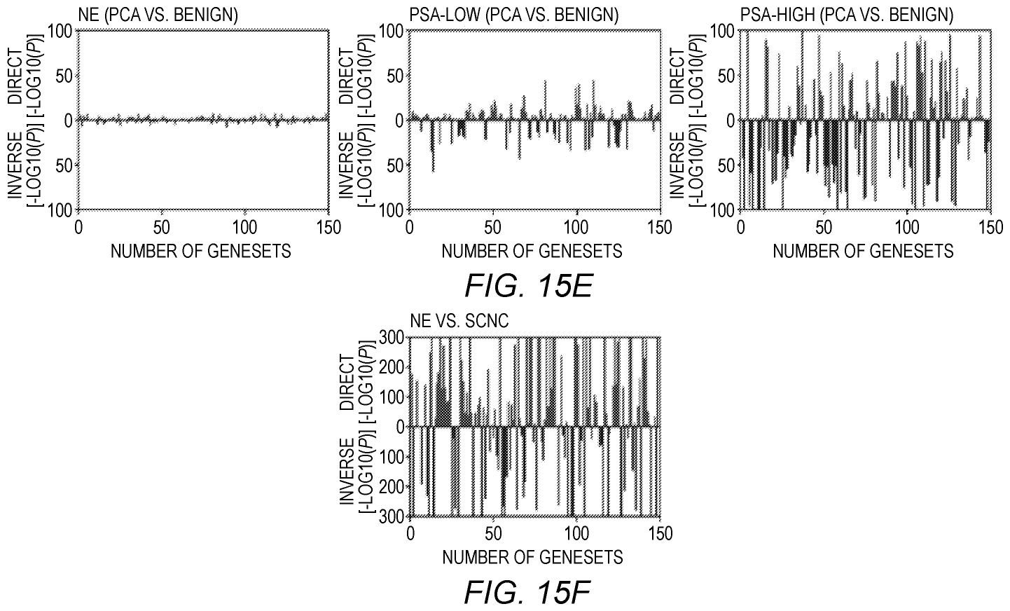

Using 897 PCa samples obtained from The Cancer Genome Atlas (TCGA) and multiple GEO data sets (Table 8), we found that the CRPCsig51 signature was significantly up-regulated in high grade, high stage, and metastatic PCa (FIG. 15A-FIG. 15F, Table 9).

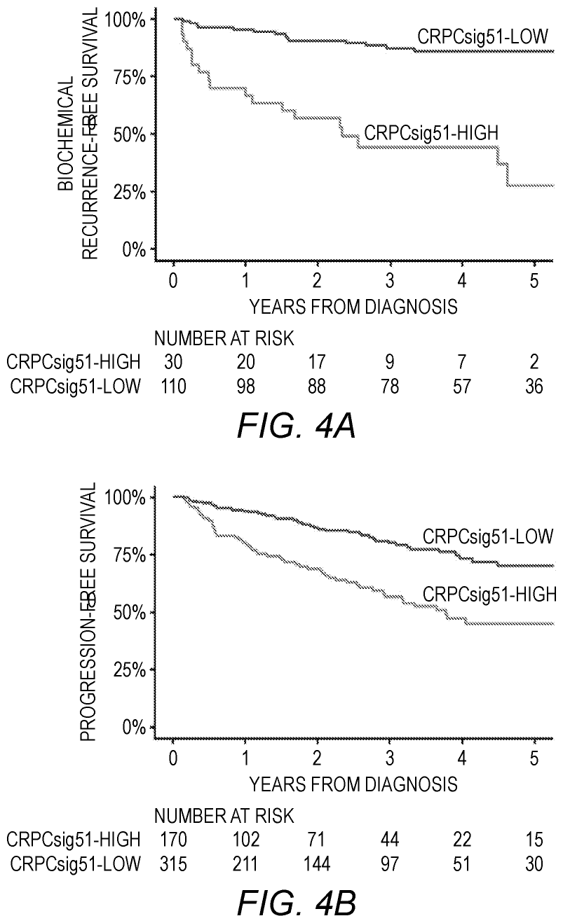

To assess the diagnostic ability of CRPCsig51 to predict survival, we used Youden's index dichotomize CRPCsig51 into low and high risk levels. A high CRPCsig51 score was associated with biochemical recurrence (GSE21034, p<0.001) and progression (TCGA, p<0.001) (FIG. 4A, 4B). When modeled as a continuous score in Cox regression, CRPCsig51 remained a significant predictor of progression (clinical recurrence and distant metastasis, or biochemical recurrence) after adjusting clinical variables, such as Gleason grade, stage, local and distant metastasis. Our results suggested that CRPC-like cells lead to disease progression independent of clinical variables of primary PCa.

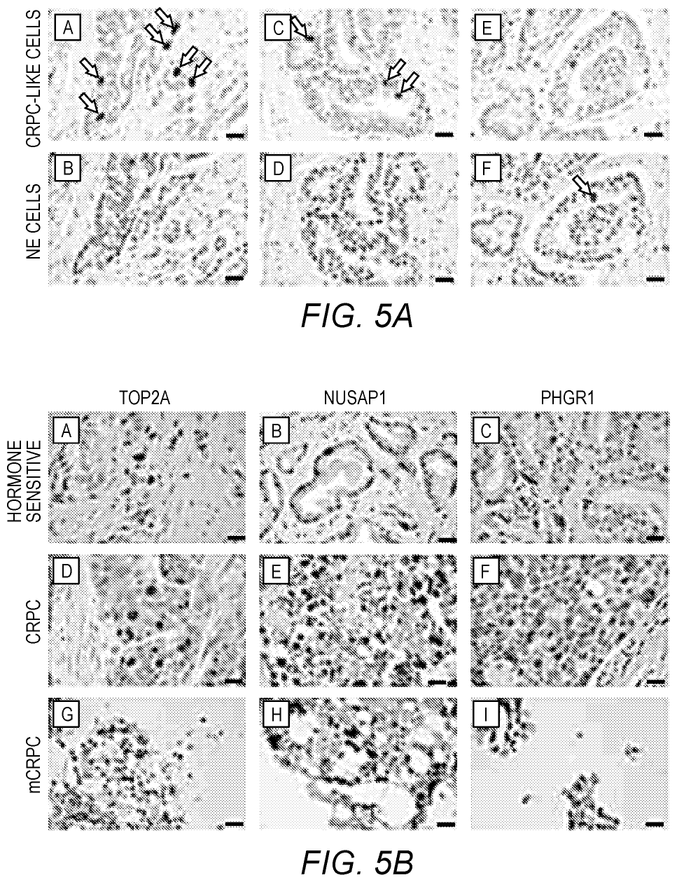

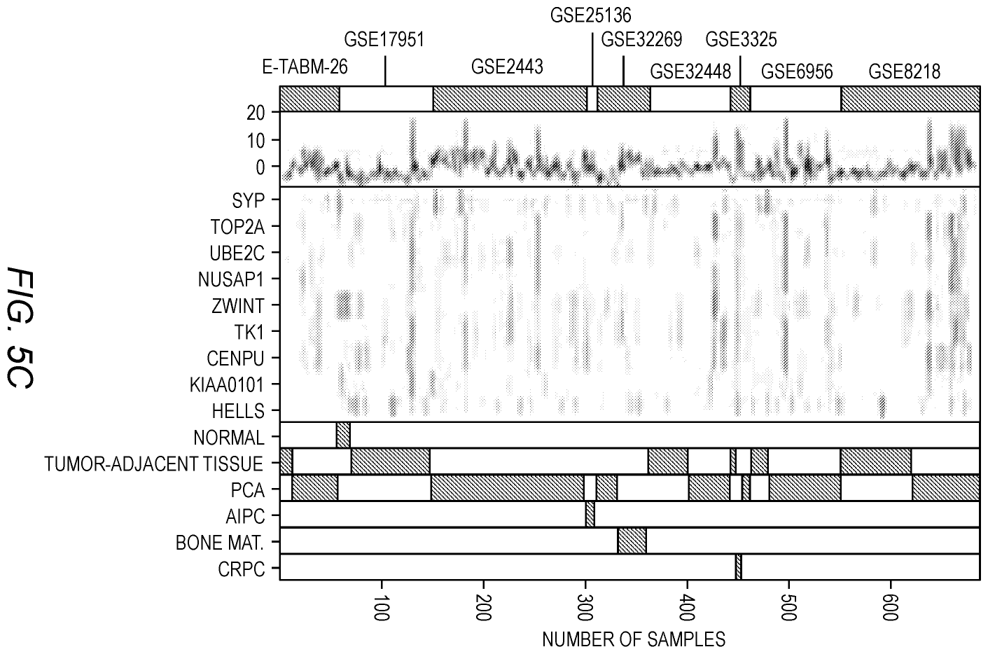

Highly plastic CRPC-like cells were observed in multiple independent datasets: To assess the pre-existing CRPC-like cells, we re-analyzed cluster specific markers using cells isolated from primary PCa samples and identified 16 markers of CRPC-like cells, including TOP2A, NUSAP1 and PHGR1. IHC analysis revealed a small fraction of CRPC-like cells in hormone sensitive PCa samples that are negative for NE marker (CHGA) (FIG. 5A). CRPC-like cells were highly enriched in CRPC (5-40%) and mCRPC samples (10-80%), which is consistent with our results using multiple public datasets (FIG. 5B). We found that 9 of 16 CRPC-like cell markers were up-regulated in both NE and CRPC-adeno clusters (FIG. 14C), and gene set enrichment analysis revealed both FOXM1 and SOX2 signaling was activated in CRPC-like cells (FIG. 14D). Unlike the cells from the CRPC-adeno sample, the C12 cells from primary PCa did not show overexpression AR, but had up-regulated HPN and EZH2, indicating that these cells were in a stage of lineage plasticity (FIG. 10B). We further validated the pre-existing CRPC-like cells using an independent cohort of 685 samples obtained from 9 datasets, and distinguished NE and CRPC-like cells using both CRPCsig51 score and SYP expression (FIG. 5C).

### C. Discussion

We have identified a subset of multipotent stem-like PSA-low luminal cells (C11) in primary PCa that can give rise to both AR-dependent and AR-independent CRPC, while CRPC-adeno can also be derived from multiple subsets of luminal cells. We identified 13 critical transcription regulators (including SOX2, AR and FOXA1) that modulate the bifurcated disease progression towards SCNC or CRPC-adeno. This molecular interrogation and sub-typing of prostate cancer cells may guide more effective therapeutic strategies.

The discovery of pre-existing CRPC-like cells in early primary PCa and in tumor-adjacent tissue represents a novel castration-resistant mechanism different from the adaptation mechanism post ADT. Although several studies combining mouse models and patient samples have previously reported the existence of CRPC-like cells during the PCa progression, isolation and characterization of these cells at single cell resolution in hormone-naïve primary human PCa has not been reported. Unlike NE cells that have the capability of self-renewal and can accelerate clonal expansion under the pressure of hormonal therapy, we found that the CRPC-like cells in primary PCa are fully developed CRPC cells, with multiple upregulated PCa progression-related oncogenes and features of highly activated lineage plasticity. Immunohistochemical stains revealed that CRPC-like cells were highly enriched in both locally recurrent and metastatic CRPC, suggesting that CRPC-like cells that are present in early PCa may emergent during androgen-deprivation as the dominant cell type.

Among the CRPC-like cell markers, TOP2A has been used to define an aggressive PCa subgroup with increased metastatic potential, and NUSAP1 expression was increased in recurrent PCa. Both TOP2A and NUSAP1 were detected in a small fraction of isolated cells within primary PCa samples. Although metastases often arise after long latency periods, early dissemination from the primary tumor can occur due to genetic diversity. Because the CRPC-like cells share identical transcriptome profile with CRPC/mCRPC, some of these cells could disseminate to distant sites prior to prostatectomy.

Using 1582 PCa and CRPC samples obtained from 14 datasets, we found that a subset of primary PCa samples is enriched with pre-exiting CRPC-like cells. To assess the prognosis of the CRPC-like cells, we developed a novel RNA expression signature (CRPCsig51) using a subset of specific markers of both NE and CRPC-like cells that were also associated with evolutionary trajectory. We found that CRPCsig51 was significantly associated with high grade and distant metastatic PCa, and the predictive power of CRPCsig51 for biochemical recurrence was independent of clinical factors. Therefore, patients whose tumors have high CRPCsig51 scores likely harbor pre-existing NE and CRPC-like cells that may become rapidly resistance to ADT, for whom early aggressive interventions may be necessary. In contrast, those patients whose tumors have low scores might be more appropriately managed by active surveillance. Future prospective studies are warranted to investigate this signature further.

### D. Conclusion

We found that a subset of prostate cancer cells with intrinsic properties of castration-resistance are present in hormone-naïve PCa before initiation of ADT. These pre-existing castration-resistant cells can rapidly expand during ADT and lead to the development of CRPC and should be sought out and targeted earlier in the evolution of prostate cancer than they are presently.

### E. Patients and Methods

Tissue collection: A total of 6 fresh primary PCa and CRPC/mCRPC specimens were collected in 2018 at Duke University Hospital, including 3 cases of primary prostate adenocarcinoma (Gleason Score 4+3, both cancer and matched tumor-adjacent tissue were procured) obtained through radical prostatectomies; 2 post-hormonal therapy locally recurrent CRPC with CRPC-adeno and SCNC histology, obtained through transurethral resection of the prostate (TURP); and 1 metastatic CRPC (mCRPC) to the soft tissue of pelvic sidewall obtained through surgical resection. A frozen section diagnosis was performed to identify cancer areas and the corresponding fresh tissue was then used for the preparation of single cells as previously described[9]. Use of anatomic materials was approved by the Duke University's Institutional Review Board.

Immunohistochemical stains (IHC) were performed using biopsies or surgically resected PCa tissue, including 18 hormone-sensitive PCa obtained through prostatectomies, 20 locally recurrent CRPC (after hormonal therapy) obtained through transurethral resection of the prostate (TURP) and 12 distant metastatic CRPC (mCRPC) cases obtained in a biopsy trial as reported previously.

Single cell RNA sequencing and data analyses: Fluorescence-activated cell sorting (FACS) was performed on single cell suspensions using flow cytometer (BD DiVa). Trop2+CD45−CXCR2− (luminal), Trop2+CD45−CD49f+ (Basal) and Trop2+CD45−CXCR2+ (NE enriched) were collected (FIG. 6), as previously described.

Suspensions of 5,000 single cells from each FACS-isolated cell collections were encapsulated into single droplets using Chromium Controller and libraries were prepared Chromium Single Cell 3′ Reagent Kits v2 (10× GENOMICS). Intra-tumor heterogeneity was delineated using Seurat, and subtype signaling was assessed using The Molecular Signatures Database (MSigDB) and ChIP-Seq data from Enrichr. Probabilistic temporal transcriptional trajectories of CRPC/SCNC progression was defined using Monocle, and validated by cluster specific mRNA signatures, as well as mutation and copy number (CNV) patterns, measured by Souporcell and HoneyBadger.

### 2. CRPC Signatures

TABLE 1
CRPCsig51 Signatures
(“1” indicates inclusion in the signature)

| Gene | CRPCsig51_old | CRPCsig51_new |
|---|---|---|
| LINC01358 | 1 | 1 |
| ASPM | 1 | 1 |
| UBE2T | 1 | 1 |
| CENPF | 1 | 1 |
| RRM2 | 1 | 1 |
| CENPU | 1 | 1 |
| CENPK | 1 | 1 |
| CENPH | 1 | 1 |
| CCDC34 | 1 | 1 |
| ZWINT | 1 | 1 |
| KIAA0101 | 1 | 1 |
| UBE2C | 1 | 1 |
| ATPIF1 | 1 | |
| CLSPN | 1 | |
| JUN | 1 | |
| DEPDC1 | 1 | |
| PSRC1 | 1 | |
| IQGAP3 | 1 | |
| NUF2 | 1 | |
| NEK2 | 1 | |
| DTL | 1 | |
| C2orf48 | 1 | |
| SPC25 | 1 | |
| PTPRN | 1 | |
| SGOL1 | 1 | |
| TACC3 | 1 | |
| MND1 | 1 | |
| CPLX2 | 1 | |
| KIF4A | 1 | |
| FABP5 | 1 | |
| MELK | 1 | |
| ZNF367 | 1 | |
| CDCA5 | 1 | |
| THYN1 | 1 | |
| SPAG6 | 1 | |
| CEP55 | 1 | |
| RAD51AP1 | 1 | |
| TROAP | 1 | |
| OTX2 | 1 | |
| RAD51 | 1 | |
| OIP5 | 1 | |
| GINS2 | 1 | |
| UBB | 1 | |
| NDC80 | 1 | |
| GRP | 1 | |
| PCSK2 | 1 | |
| CST4 | 1 | |
| MYBL2 | 1 | |
| SYT5 | 1 | |
| RIBC2 | 1 | |
| GTSE1 | 1 | |
| ITGB3BP | | 1 |
| LINC00869 | | 1 |
| BARD1 | | 1 |
| DTYMK | | 1 |
| CCDC14 | | 1 |
| RBP1 | | 1 |
| SLC4A4 | | 1 |
| MAD2L1 | | 1 |
| CCNB1 | | 1 |
| LMNB1 | | 1 |
| CENPW | | 1 |
| SYP | | 1 |
| GGH | | 1 |
| CEP78 | | 1 |
| SMC2 | | 1 |
| PHF19 | | 1 |
| GLYATL1 | | 1 |
| FAM111B | | 1 |
| FEN1 | | 1 |
| CADM1 | | 1 |
| CDK1 | | 1 |
| HELLS | | 1 |
| MKI67 | | 1 |
| TMPO | | 1 |
| C1QTNF9B-AS1 | | 1 |
| CKAP2 | | 1 |
| CDKN3 | | 1 |
| NUSAP1 | | 1 |
| PRC1 | | 1 |
| ATAD5 | | 1 |
| TOP2A | | 1 |
| TK1 | | 1 |
| BIRC5 | | 1 |
| TYMS | | 1 |
| TPX2 | | 1 |
| RNASEH2A | | 1 |
| ASF1B | | 1 |
| ISYNA1 | | 1 |
| TFF3 | | 1 |

TABLE 2
CRPCsig51 Signature Gene Names (CRPCsig51_new)

| Gene Symbol | Full Gene Name |
|---|---|
| ASF1B | anti-silencing function 1B histone chaperone |
| ASPM | assembly factor for spindle microtubules |
| ATAD5 | ATPase family AAA domain containing 5 |
| BARD1 | BRCA1 associated RING domain 1 |
| BIRC5 | baculoviral IAP repeat containing 5 |
| CADM1 | cell adhesion molecule 1 |
| CCDC14 | coiled-coil domain containing 14 |
| CCDC34 | coiled-coil domain containing 34 |
| CCNB1 | cyclin B1 |
| CDK1 | cyclin dependent kinase 1 |
| CDKN3 | cyclin dependent kinase inhibitor 3 |
| CENPF | centromere protein F |
| CENPH | centromere protein H |
| CENPK | centromere protein K |
| CENPU | centromere protein U |
| CENPW | centromere protein W |
| CEP78 | centrosomal protein 78 |
| CKAP2 | cytoskeleton associated protein 2 |
| DTYMK | deoxythymidylate kinase |
| FAM111B | FAM111 trypsin like peptidase B |
| FEN1 | flap structure-specific endonuclease 1 |
| GGH | gamma-glutamyl hydrolase |
| GLYATL1 | glycine-N-acyltransferase like 1 |
| HELLS | helicase, lymphoid specific |
| ISYNA1 | inositol-3-phosphate synthase 1 |
| ITGB3BP | integrin subunit beta 3 binding protein |
| LINC00869 | long intergenic non-protein coding RNA 869 |
| LINC01358 | long intergenic non-protein coding RNA 1358 |
| LMNB1 | lamin B1 |
| MAD2L1 | mitotic arrest deficient 2 like 1 |
| MKI67 | marker of proliferation Ki-67 |
| NUSAP1 | nucleolar and spindle associated protein 1 |
| KIAA0101 | PCNA clamp associated factor |
| C1QTNF9B- | prostate and testis expressed opposite C1QTNF9B |
| AS1 | and MIPEP |
| PHF19 | PHD finger protein 19 |
| PRC1 | protein regulator of cytokinesis 1 |
| RBP1 | retinol binding protein 1 |
| RNASEH2A | ribonuclease H2 subunit A |
| RRM2 | ribonucleotide reductase regulatory subunit M2 |
| SLC4A4 | solute carrier family 4 member 4 |
| SMC2 | structural maintenance of chromosomes 2 |
| SYP | synaptophysin |
| TFF3 | trefoil factor 3 |
| TK1 | thymidine kinase 1 |
| TMPO | thymopoietin |
| TOP2A | DNA topoisomerase II alpha |
| TPX2 | TPX2 microtubule nucleation factor |
| TYMS | thymidylate synthetase |
| UBE2C | ubiquitin conjugating enzyme E2 C |
| UBE2T | ubiquitin conjugating enzyme E2 T |
| ZWINT | ZW10 interacting kinetochore protein |

TABLE 3
CRPCsig51 Signature Gene Identifiers

| Gene | | | NCBI Gene | Accession | |
|---|---|---|---|---|---|
| Symbol | HGNC_ID | Ensembl_ID | ID | numbers | RefSeq_ID |
| ASF1B | 20996 | ENSG00000105011 | 55723 | AF279307 | NM_018154 |
| ASPM | 19048 | ENSG00000066279 | 259266 | AY367065 | NM_018136 |
| ATAD5 | 25752 | ENSG00000176208 | 79915 | | NM_024857 |
| BARD1 | 952 | ENSG00000138376 | 580 | | NM_000465 |
| BIRC5 | 593 | ENSG00000089685 | 332 | U75285 | NM_001168 |
| CADM1 | 5951 | ENSG00000182985 | 23705 | AB017563 | NM_014333 |
| CCDC14 | 25766 | ENSG00000175455 | 64770 | AL122079 | NM_022757 |
| CCDC34 | 25079 | ENSG00000109881 | 91057 | AF382034 | NM_030771 |
| CCNB1 | 1579 | ENSG00000134057 | 891 | U22364 | NM_031966 |
| CDK1 | 1722 | ENSG00000170312 | 983 | BC014563 | NM_001786 |
| CDKN3 | 1791 | ENSG00000100526 | 1033 | U02681 | NM_001130851 |
| CENPF | 1857 | ENSG00000117724 | 1063 | U30872 | NM_016343 |
| CENPH | 17268 | ENSG00000153044 | 64946 | AB035124 | NM_022909 |
| CENPK | 29479 | ENSG00000123219 | 64105 | BC008504 | NM_022145 |
| CENPU | 21348 | ENSG00000151725 | 79682 | AK027121, | NM_024629 |
| CENPW | 21488 | ENSG00000203760 | | AF516710 | |
| CEP78 | 25740 | ENSG00000148019 | 387103 | BC039556 | NM_001012507 |
| CKAP2 | 1990 | ENSG00000136108 | 84131 | BC058931 | NM_001098802 |
| DTYMK | 3061 | ENSG00000168393 | 26586 | AF177227 | NM_001098525 |
| FAM111B | 24200 | ENSG00000189057 | 1841 | X54729 | NM_012145 |
| FEN1 | 3650 | ENSG00000168496 | 374393 | BC062456 | NM_198947 |
| GGH | 4248 | ENSG00000137563 | 2237 | L37374 | NM_004111 |
| GLYATL1 | 30519 | ENSG00000166840 | 8836 | U55206 | NM_001410926 |
| HELLS | 4861 | ENSG00000119969 | 92292 | AK091965 | NM_080661 |
| ISYNA1 | 29821 | ENSG00000105655 | 3070 | AF155827 | NM_018063 |
| ITGB3BP | 6157 | ENSG00000142856 | 51477 | | NM_016368 |
| LINC00869 | 29050 | ENSG00000277147 | 23421 | U37139 | NM_014288 |
| LINC01358 | 50589 | ENSG00000237352 | 57234 | AB007962 | NR_046135 |
| LMNB1 | 6637 | ENSG00000113368 | 101926925 | | NR_110626 |
| MAD2L1 | 6763 | ENSG00000164109 | 4001 | L37737 | NM_005573 |
| MKI67 | 7107 | ENSG00000148773 | 4085 | U65410 | NM_002358 |
| NUSAP1 | 18538 | ENSG00000137804 | 4288 | X65550 | NM_002417 |
| KIAA0101 | 28961 | ENSG00000166803 | 51203 | AF290612 | NM_016359 |
| C1QTNF9B- | 39839 | ENSG00000205861 | 9768 | D14657 | NM_014736 |
| AS1 | | | 542767 | | NR_172510 |
| PHF19 | 24566 | ENSG00000119403 | 26147 | BX640713 | NM_001286840 |
| PRC1 | 9341 | ENSG00000198901 | 9055 | AF044588 | NM_003981 |
| RBP1 | 9919 | ENSG00000114115 | 5947 | | NM_002899 |
| RNASEH2A | 18518 | ENSG00000104889 | 10535 | Z97029 | NM_006397 |
| RRM2 | 10452 | ENSG00000171848 | 6241 | | NM_001034 |
| SLC4A4 | 11030 | ENSG00000080493 | 8671 | AF007216 | NM_003759 |
| SMC2 | 14011 | ENSG00000136824 | 10592 | AF092563 | NM_001042550 |
| SYP | 11506 | ENSG00000102003 | 6855 | X06389 | NM_003179 |
| TFF3 | 11757 | ENSG00000160180 | 7033 | AF432265 | NM_003226 |
| TK1 | 11830 | ENSG00000167900 | 7083 | | NM_003258 |
| TMPO | 11875 | ENSG00000120802 | 7112 | | NM_003276 |
| TOP2A | 11989 | ENSG00000131747 | 7153 | | NM_001067 |
| TPX2 | 1249 | ENSG00000088325 | 22974 | AF098158 | NM_012112 |
| TYMS | 12441 | ENSG00000176890 | 7298 | X02308 | NM_001071 |
| UBE2C | 15937 | ENSG00000175063 | 11065 | U73379 | NM_007019 |
| UBE2T | 25009 | ENSG00000077152 | 29089 | AF161499 | NM_014176 |
| ZWINT | 13195 | ENSG00000122952 | 11130 | AF067656 | NM_001005413 |

TABLE 4
CRPCsig51_old and _new Signature Overlapping Genes

| Gene Symbol | Full Gene Name |
|---|---|
| ASPM | assembly factor for spindle microtubules |
| CCDC34 | coiled-coil domain containing 34 |
| CENPF | centromere protein F |
| CENPH | centromere protein H |
| CENPK | centromere protein K |
| CENPU | centromere protein U |
| LINC01358 | long intergenic non-protein coding RNA 1358 |
| KIAA0101 | PCNA clamp associated factor |
| RRM2 | ribonucleotide reductase regulatory subunit M2 |
| UBE2C | ubiquitin conjugating enzyme E2 C |
| UBE2T | ubiquitin conjugating enzyme E2 T |
| ZWINT | ZW10 interacting kinetochore protein |

### 3. Cellular Heterogeneity of Prostate Cancer Contributes to Therapy Resistance and Disease Progression

### A. Materials and Methods

Tissue collection: In total of 6 fresh primary PCa and CRPC/mCRPC specimen were collected in 2018 at Duke University Hospital. These samples included: 3 primary prostate adenocarcinoma (Gleason Score 4+3, both cancer and matched benign prostate tissue) obtained through radical prostatectomy; 2 locally recurrent CRPC with either CRPC-adeno or SCNC histology obtained through transurethral resection of the prostate (TURP); and 1 metastatic CRPC (mCRPC) to pelvic sidewall obtained through surgical resection. For the prostatectomy specimen, the fresh prostate was received in pathology, weighed, measured, inked and sectioned. A frozen section diagnosis was performed to identify benign and cancer areas and the corresponding fresh tissue was then used for the preparation of single cells as previously described. For locally recurrent and mCRPC specimens, a portion of the resected fresh tissue was submitted for research. Fresh tissue chunks were used directly for single cell preparation as previously described.

Duke Biorepository and Precision Pathology Core functioned as the honest broker, and researchers received de-identified samples that cannot be traced to specific patients. Use of anatomic materials was approved by the Duke institutional Review Board.

Fluorescence-Activated Cell Sorting: Different subsets of prostate epithelial cells were selected by flow cytometric cell sorting (FACS), as previously described. Trop2+CD45− cells were collected from single cell suspensions. Based on our previous work demonstrating that NE cells in PCa overexpress IL8 receptor CXCR2, an anti-CXCR2 antibody (eBio5E8-C7-F10) has been used to isolate NE cells with consistent success. Before cell sorting, primary prostate cancer cells were re-suspended in ADMEM/F12 containing 1×HEPES and 1× Glutamax and incubated with antibodies against CD45, Trop2, CD49f, and CXCR2 for 30 min at 4° C. FACS was performed on single cell suspensions using flow cytometer (BD DiVa). Trop2+CD45−CXCR2+ (NE enriched), Trop2+CD45−CXCR2− (luminal) were collected from malignant tissue of primary PCa, locally recurrent and metastatic cancers. Trop2+CD45−CD49f+ (Basal), Trop2+CD54−CXCR2+ (NE enriched), Trop2+CD45−CD49f−CXCR2− (luminal) were collected from benign prostate tissue from the prostatectomy specimen. The purity of the sorted populations was verified by flow cytometry.

Single cell library construction, sequencing and pre-processing of seRNA-seq data: The suspension of 5,000 single cells from each FACS-isolated cell collections were encapsulated into single droplets using Chromium Controller (10× GENOMICS) and libraries were prepared using Chromium Single Cell 3′ Reagent Kits v2 (10× GENOMICS) according to manufacturer's protocols (10× Genomics, CG00052). The final libraries from each experiment were sequenced on NovaSeq 6000 S2 as 150-bp paired-end reads in the NantOmics LLC, Culver City CA; or NovaSeq 6000 S1 as 100-bp paired-end reads in GCB Sequencing and Genomic Technologies Core at Duke University. Sequencing results of individual library were run through CellRanger mkfastq (10× GENOMICS, version 2.2.0) for demultiplexing, barcode processing and converting to fastq format. The fastq files were grouped by sample (across sequencing runs) and GRCh38 was used as a reference to obtain bam alignment files. Libraries that passed QC were pooled by running through CellRanger count to obtain unique molecular identifiers (UMIs) matrices. Multiple single cell libraries were combined using Cellranger aggr through deep normalization, in order to avoid the batch effect introduced by sequencing depth. 28,362 single cell libraries were successfully sequenced, with deep coverage reads of 81,393 Post-Normalization Mean Reads per Cell with 2,673 Median Genes per Cell and 10,679 median UMI counts per cell (FIG. 6A, 6B). Using CellRanger normalized raw expression counts of the genes that expressed over 95% of cells, we performed principal component analysis, and no significant batch effect was observed (FIG. 7A).

Cell clustering, Visualization and Finding differentially expressed features: Further analysis, including quality filtering, the identification of highly variable genes, normalization, dimensionality reduction, standard unsupervised clustering algorithms and the discovery of differentially expressed genes, was performed using the Seurat R package (ver. 2.3.4). To remove doublets and poor-quality cells, cells were excluded from subsequent analysis if they expressed less than 500 genes or expressed over 8000 genes, and if the percentage of mitochondrial genes was greater than 10% per cell. After removing unwanted cells from the dataset, we normalized the data by the total expression, multiplied by a scale factor of 10,000 and log-transformed the result. In total, 24,385 single cells (16,876 epithelium cells from primary PCa and 7,509 CRPC epithelium cells) were selected for the subsequence analyses. Primary sources of heterogeneity in the dataset (scared expression data) before clustering were visualized using PCA plot, and no significant batch effect was observed (FIG. 7B).

Using the top 10 calculated principal components and a resolution of 0.6, we performed both t-Distributed Stochastic Neighbor Embedding (t-SNE) and Uniform Manifold Approximation and Projection (UMAP) (Seurat ver. 3.1) for non-linear dimension-reduction and visualization. Though similar subpopulation structure was observed, t-SNE plot showed a better separation of individual clusters (FIG. 7C to FIG. 7F). In total, 17 transcriptionally similar cell subpopulations were identified. To find markers of individual cluster, we performed pairwise differential expression analysis (Wilcox test in Seurat) by adjusting for cellular detection rate for each cluster against all other clusters that were detected in at least 25% of the cluster cells, keeping the positive markers that were significant (Bonferroni adjusted P<0.01) in each of the comparisons. The cluster specific markers were further selected if the average expression of marker was 2-fold higher than the average expression of any other individual clusters. In total, 1935 cluster specific markers were identified, and the top 10 markers of each cluster were plotted on headmap to feature differences of the cell types (FIG. 8A).

To determine if these cell clusters indeed correspond to distinct cellular lineage classes, visualized the distribution of cells isolated from either primary PCa or CRPC/SCNC samples (FIG. 9A-FIG. 9F), and measure the expression of well-established basal, luminal and NE markers by displaying the relative expression of each gene on a distributed Stochastic Neighbor Embedding (tSNE) plot (FIG. 1E, FIG. 10A, 10B).

Signature-based cell classification: In total of 10797 of molecular signatures were obtained from The Molecular Signatures Database (MSigDB), including Hallmark gene sets (n=50), oncogenic signatures (n=99), curated gene sets (n=4731) and GO gene sets (n=5917) were downloaded from Molecular Signatures Database (MSigDBv6.2) MSigDB hallmark gene sets were used to access well-defined biological states or processes, and de-regulated cellular pathways in cancer cells were defined using MSigDB oncogenic gene sets. For oncogenic signatures, only up-regulated gene sets were selected (n=99). Principal component analyses using Seurat scaled gene expression data of all 24,385 cells was performed, and used first principal component (PC1) to assign each of the cells with a gene set signature score.

The average gene set PC1 score of 45 gene sets that are up-regulated in a wide variety of stem cells obtained from Curated Gene Sets and GO Gene Sets was used to assess stem-cell features (Table 6). The average gene set PC1 score of 36 gene sets that are up-regulated in a wide variety of cell proliferative process obtained from Curated Gene Sets and GO Gene Sets was used to build a proliferation signature (Table 7). The TP53 deficient signaling was assessed using average score of oncogenic gene sets “P53_DN.V1_UP” and “P53_DN.V2_UP”. The RB inactivation was assessed using average score of oncogenic gene sets “RB_DN.V1_UP” and “RB P107_DN.V1_UP”. Because a combined gene signature was used to assign each cell a probability of stem-cell features, TP53 deficient signal and RB inactivation, there is no absolute cut off for the classification of true stem cells. Therefore, we used top 25% quartile signature scores to define highest probability of stem cells and bottom 25% quartile signature scores to define lowest probability of stem cells (FIG. 2B).

AR response signature and EMT signature were obtained Hallmark gene sets. SCNC signature, MDCS signature and IBC resistance signature were built by using first principal component (PC1) of gene set published by Beltran et al or Jiang et al. Evolutionary signature of CRPC progression (CRPCsig51), luminal lineage and basal lineage signatures (Table 5) were also built by principal component analysis. To assess the risk of prostate cancer recurrence, we subdivided primary PCa samples into high-risk (top 25% quartile highest signature scores), intermediate-risk (middle 50% quartile signature scores), and low-risk (bottom 25% quartile lowest signature scores) of recurrent groups according the CRPCsig51 signature score.

Single-Cell Trajectory Reconstruction: To analyze CRPC progression across multiple developmental stages, single-cell pseudotime trajectories were constructed with Monocle (version 2.6.4). Using Reversed Graph Embedding, single cells were projected onto a manifold in a low-dimensional space, which orders them into a trajectory and identifies any branch points corresponding to cell fate decisions. The dataset was subset to only include either primary PCa cells or CRPC cells without basal cell clusters, or mixed PCa and CRPC cells without basal cell clusters, for the analyses. Genes that differ between the clusters on the basis of a likelihood ratio test between a generalized linear model using Seurat defined cell clusters were identified. The top 8,000 significantly differentially expressed genes were selected as the ordering genes for the trajectory reconstruction. Expression profiles were reduced to 2 dimensions using the DDRTree algorithm included with Monocle-2, via the reduce Dimension with 4-10 components.

Evolutionary analyses: To trace the subpopulations of cells in primary PCa that have higher risks of progressing to CRPC, correlation among the identified cell clusters were analyzed using cluster specific mRNA signature, mutation and copy number (CNV) patterns of CRPC cells. First, a series of principal component analyses was performed to build cluster specific signatures using identified cluster specific markers. Each of the 24,385 cells were assigned a cluster specific signature score and determine the clonal connection between primary PCa and CRPC by testing if CRPC cell clusters (C3, C8 and C10) carried any PCa cluster specific signature using receiver operating characteristic (ROC) analysis (FIG. 3C to FIG. 3D). Because the limitation of current single-cell sequencing technology that cannot measure both RNA expression and mutation or CNV on same cells, HoneyBadger and Souporcell algorithms were therefore applied to process CNV and genotype (SNP+mutation) patterns of each CRPC sample using scRNA-sequencing data.

In considering the effect genetic background of cells isolated from different patient, CNV and mutation genotyping was processed use the cells isolated from same CRPC patient. Souporcell was used to read genotype variance (both SNP and mutation) and allele counts of each cell from CellRanger output, and defined 11 genotype clusters from each CRPC sample. The scores genotype clusters were further applied for a hierarchical clustering (centroid linkage), and the dendrogram was used to visualize the hierarchical relationship among the cells with singleton genotype. (FIG. 3E, FIG. FIG. 14A, 14B). HoneyBadger was used to measure the CNV events in single cells and reconstructed subclonal architecture using either allele or expression information from CellRanger output of single-cell RNA-sequencing data. Using common heterozygous SNPs from “ExAC.0.3.GRCh38.vcf.gz” obtained from ExAC database, the number of reads corresponding to each SNP site for each cell was measured using bam files from CellRanger output, and determined persistent allelic imbalance (detected from putative heterozygous variants) to identify LOH events. Because limited allele counts can be assessed using HoneyBadger allele model, multiple LOH events could only be identified from CRPC1 sample (the local recent CRPC sample) (FIG. 3F). After removed the cells with LOH probability less than 25%, the relationship among LOH events was assessed by hierarchical clustering. The LOH presenting cells was defined as the cells who showed LOH probability great than 75%.

The HoneyBadger expression model requires gene expression matrices for tumor cells along with the expression reference from matched normal cells. Since basal cells are non-malignant cells in prostate tumor, the average expression of basal cells isolated from same sample was used as normal reference Using HoneyBadger expression model, multiple CNV events was successfully mesured from CRPC2 sample (the local recurrent CRPC with SCNC histology) (FIG. 14C). After removed the cells with CNV probability less than 25%, the relationship among the CNV events was assessed by hierarchical clustering. The CNV presenting cells was defined as the cells who showed CNV probability great than 65%. The temporal relationship was assessed among the cell subpopulations by clustering the genotyping or CNV patterns (FIG. 3E, 3F, FIG. 14A-FIG. 14D), and determine if any evolutionary connection observed by Monocle trajectories can be confirmed by at least two of the other tests, including the signature ROC test, genotyping pattern test and CNV clustering test.

Pathway enrichment analysis: Hypergeometric test for enrichment analysis (R package) was performed to assess the enrichment of Hallmark gene sets and oncogenic signatures obtained from Molecular Signatures Database (MSigDB v6.2). ChIP-X enrichment analysis (ChEA) was performed using Enrichr. Gene set signature clustering was performed using Cluster 3.0.

Gene correlation analysis: Seurat scaled expression matrix was used for Gene-correlation analyses. Differentially expressed genes between primary PCa cells and CRPC subtypes were determined using Mann-Whitney U Test (MWU), and genes upregulated in CRPC cells were selected using a significance cutoff at Bonferroni adjusted P<0.01. CRPC evolutionary trajectory associated genes were determined by linear regression testing for pseudotime with a cutoff for significance at Bonferroni adjusted P<0.01. Statistical analyses were performed using the software: R Project for Statistical Computing, Matlab, STATISTICA, PRISM program (GraphPad), Gene Cluster 3.0 and Java TreeView. B. Tables 5 through 9

TABLE 5
Lineage signature genes

| Gene | Signature |
|---|---|
| AR | Luminal lineage signature |
| KLK3 | Luminal lineage signature |
| FOXA1 | Luminal lineage signature |
| HOXB13 | Luminal lineage signature |
| TMPRSS2 | Luminal lineage signature |
| KRT8 | Luminal lineage signature |
| CDH1 | Luminal lineage signature |
| TP63 | Basal signature |
| KRT5 | Basal signature |
| KRT6A | Basal signature |
| ITGA6 | Basal signature |

TABLE 6

Stem-cell related gene sets
GO_NEURONAL_STEM_CELL_DIVISION
GO_NEURONAL_STEM_CELL_POPULATION_MAINTENANCE
GO_SOMATIC_STEM_CELL_POPULATION_MAINTENANCE
GO_NEGATIVE_REGULATION_OF_STEM_CELL_DIFFERENTIATION
GO_STEM_CELL_PROLIFERATION
GO_HEMATOPOIETIC_STEM_CELL_DIFFERENTIATION
GO_NEGATIVE_REGULATION_OF_STEM_CELL_PROLIFERATION
GO_STEM_CELL_DIVISION
GO_HEMATOPOIETIC_STEM_CELL_PROLIFERATION
GO_POSITIVE_REGULATION_OF_STEM_CELL_DIFFERENTIATION
GO_REGULATION_OF_STEM_CELL_POPULATION_MAINTENANCE
GO_REGULATION_OF_STEM_CELL_PROLIFERATION
GO_SOMATIC_STEM_CELL_DIVISION
GO_STEM_CELL_DIFFERENTIATION
GO_POSITIVE_REGULATION_OF_STEM_CELL_PROLIFERATION
GO_REGULATION_OF_STEM_CELL_DIFFERENTIATION
GAL_LEUKEMIC_STEM_CELL_UP
OSWALD_HEMATOPOIETIC_STEM_CELL_IN_COLLAGEN_GEL_UP
HOEBEKE_LYMPHOID_STEM_CELL_UP
LEE_NEURAL_CREST_STEM_CELL_UP
JAATINEN_HEMATOPOIETIC_STEM_CELL_UP
LIANG_HEMATOPOIESIS_STEM_CELL_NUMBER_LARGE_VS_TINY_UP
LIANG_HEMATOPOIESIS_STEM_CELL_NUMBER_SMALL_VS_HUGE_UP
BYSTRYKH_HEMATOPOIESIS_STEM_CELL_FGF3
BYSTRYKH_HEMATOPOIESIS_STEM_CELL_QTL_CIS
BYSTRYKH_HEMATOPOIESIS_STEM_CELL_AND_BRAIN_QTL_TRANS
BYSTRYKH_HEMATOPOIESIS_STEM_CELL_AND_BRAIN_QTL_CIS
BYSTRYKH_HEMATOPOIESIS_STEM_CELL_RUNX1
BYSTRYKH_HEMATOPOIESIS_STEM_CELL_QTL_TRANS
BYSTRYKH_HEMATOPOIESIS_STEM_CELL_FLI1
IVANOVA_HEMATOPOIESIS_STEM_CELL_LONG_TERM
IVANOVA_HEMATOPOIESIS_STEM_CELL_SHORT_TERM
IVANOVA_HEMATOPOIESIS_STEM_CELL_AND_PROGENITOR
IZADPANAH_STEM_CELL_ADIPOSE_VS_BONE_UP
BOQUEST_STEM_CELL_UP
BOQUEST_STEM_CELL_CULTURED_VS_FRESH_UP
BEIER_GLIOMA_STEM_CELL_UP
YAMASHITA_LIVER_CANCER_STEM_CELL_UP
LIANG_HEMATOPOIESIS_STEM_CELL_NUMBER_QTL
BYSTRYKH_HEMATOPOIESIS_STEM_CELL_IL3RA
BYSTRYKH_HEMATOPOIESIS_STEM_CELL_SCP2_QTL_TRANS
WONG_EMBRYONIC_STEM_CELL_CORE
PECE_MAMMARY_STEM_CELL_UP
GENTLES_LEUKEMIC_STEM_CELL_UP
LIM_MAMMARY_STEM_CELL_UP

TABLE 7
Proliferation related gene sets

GO_REGULATION_OF_HEPATOCYTE_PROLIFERATION
GO_REGULATION_OF_LEUKOCYTE_PROLIFERATION
GO_B_CELL_PROLIFERATION
GO_POSITIVE_REGULATION_OF_B_CELL_PROLIFERATION
GO_MUSCLE_CELL_PROLIFERATION
GO_POSITIVE_REGULATION_OF_EPITHELIAL_CELL_PROLIFERATION
GO_REGULATION_OF_ACTIVATED_T_CELL_PROLIFERATION
GO_POSITIVE_REGULATION_OF_NEUROBLAST_PROLIFERATION
GO_STEM_CELL_PROLIFERATION
GO_POSITIVE_REGULATION_OF_LEUKOCYTE_PROLIFERATION
GO_INNER_CELL_MASS_CELL_PROLIFERATION
GO_REGULATION_OF_CELL_PROLIFERATION_INVOLVED_IN_KIDNEY_DEVELOPMENT
GO_REGULATION_OF_GLIAL_CELL_PROLIFERATION
GO_HEMATOPOIETIC_STEM_CELL_PROLIFERATION
GO_POSITIVE_REGULATION_OF_ALPHA_BETA_T_CELL_PROLIFERATION
GO_REGULATION_OF_OSTEOBLAST_PROLIFERATION
GO_REGULATION_OF_T_CELL_PROLIFERATION
GO_REGULATION_OF_NEUROBLAST_PROLIFERATION
GO_REGULATION_OF_SMOOTH_MUSCLE_CELL_PROLIFERATION
GO_ENDOTHELIAL_CELL_PROLIFERATION
GO_POSITIVE_REGULATION_OF__ACTIVATED_T_CELL_PROLIFERATION
GO_REGULATION_OF_B_CELL_PROLIFERATION
GO_REGULATION_OF_CELL_PROLIFERATION_INVOLVED_IN_HEART_MORPHOGENESIS
GO_POSITIVE_REGULATION_OF_MESENCHYMAL_CELL_PROLIFERATION
GO_POSITIVE_REGULATION_OF_STEM_CELL_PROLIFERATION
GO_REGULATION_OF_NEURAL_PRECURSOR_CELL_PROLIFERATION
GO_REGULATION_OF_MYOBLAST_PROLIFERATION
GO_REGULATION_OF_CELL_PROLIFERATION
GO_REGULATION_OF_SKELETAL_MUSCLE_CELL_PROLIFERATION
ROSTY_CERVICAL_CANCER_PROLIFERATION_CLUSTER
BENPORATH_PROLIFERATION
GOLDRATH_HOMEOSTATIC_PROLIFERATION
PEART_HDAC_PROLIFERATION_CLUSTER_UP
FIRESTEIN_PROLIFERATION
CHIANG_LIVER_CANCER_SUBCLASS_PROLIFERATION_UP
ACOSTA_PROLIFERATION_INDEPENDENT_MYC_TARGETS_UP

TABLE 8
Primary PCa tumor enriched gene sets

| | Enrichment score | | | | | | |
|---|---|---|---|---|---|---|---|
| Term | C5 | C11 | C13 | C15 | C12 | C14 | C4 |
| FOXA1 21915096 ChIP-Seq LNCaP-1F5 Human | 3.9 | 25.6 | 43.1 | 44.4 | 50.1 | 44.5 | 24.8 |
| AR 21915096 ChIP-Seq LNCaP-1F5 Human | 1.5 | 49.1 | 28.0 | 6.6 | 13.1 | 20.7 | 34.7 |
| SOX2 20726797 ChIP-Seq SW620 Human | 31.0 | 110.3 | 10.2 | 3.9 | 51.8 | 13.5 | 22.2 |
| RELA 24523406 ChIP-Seq FIBROSARCOMA Human | 25.7 | 46.0 | 11.2 | 17.3 | 23.8 | 17.5 | 7.5 |
| ESR2 21235772 ChIP-Seq MCF-7 Human | 11.4 | 38.6 | 59.1 | 32.8 | 8.8 | 16.6 | 3.1 |
| FOXM1 26456572 ChIP-Seq MCF-7 Human | 0.5 | 36.1 | 8.9 | 6.6 | 8.2 | 1.3 | 3.3 |
| ESR1 21235772 ChIP-Seq MCF-7 Human | 5.8 | 25.8 | 56.8 | 17.0 | 24.0 | 0.0 | 0.7 |
| SMAD2 18955504 ChIP-ChIP HaCaT Human | 1.8 | 28.2 | 9.5 | 7.0 | 68.4 | 7.7 | 14.9 |
| SMAD3 18955504 ChIP-ChIP HaCaT Human | 1.8 | 28.2 | 9.5 | 7.0 | 68.4 | 7.7 | 14.9 |
| SMAD4 19686287 ChIP-ChIP HaCaT Human | 7.0 | 35.0 | 0.0 | 0.0 | 25.5 | 0.0 | 1.1 |
| GATA4 25053715 ChIP-Seq YYC3 Human | 6.3 | 28.0 | 16.7 | 2.2 | 19.5 | 20.7 | 6.4 |
| SMAD 19615063 ChIP-ChIP OVARY Human | 3.1 | 27.2 | 0.0 | 0.0 | 12.5 | 0.0 | 0.0 |
| ZNF263 19887448 ChIP-Seq K562 Human | 0.6 | 25.7 | 0.0 | 0.0 | 0.0 | 0.0 | 5.7 |
| JUN 26020271 ChIP-Seq SMOOTH MUSCLE Human | 2.7 | 25.6 | 0.2 | 0.0 | 8.2 | 1.3 | 6.4 |
| JUND 26020271 ChIP-Seq SMOOTH MUSCLE Human | 5.3 | 23.3 | 0.2 | 0.0 | 8.2 | 7.2 | 10.8 |
| AHR 22903824 ChIP-Seq MCF-7 Human | 3.7 | 21.9 | 10.3 | 3.0 | 3.6 | 0.0 | 0.9 |
| ESR1 15608294 ChIP-ChIP MCF-7 Human | 32.5 | 2.4 | 39.5 | 54.6 | 17.6 | 0.0 | 3.3 |
| GATA3 24758297 ChIP-Seq MCF-7 Human | 5.3 | 10.8 | 62.7 | 26.8 | 81.5 | 20.7 | 4.7 |
| HIF1A 21447827 ChIP-Seq MCF-7 Human | 6.1 | 3.5 | 88.6 | 49.3 | 36.8 | 24.7 | 0.3 |
| RACK7 27058665 Chip-Seq MCF-7 Human | 6.1 | 13.8 | 28.0 | 6.6 | 19.5 | 7.2 | 1.5 |
| PIAS1 25552417 ChIP-Seq VCAP Human | 2.7 | 14.7 | 24.4 | 13.5 | 3.0 | 7.1 | 3.6 |
| FOXO3 23340844 ChIP-Seq DLD1 Human | 8.0 | 14.7 | 27.6 | 15.3 | 37.5 | 7.9 | 0.3 |
| FOXA1 27197147 Chip-Seq ENDOMETRIOID- | 7.8 | 2.3 | 35.4 | 19.6 | 27.6 | 10.1 | 16.6 |
| ADENOCARCINOMA Human | | | | | | | |
| TRIM28 21343339 ChIP-Seq HEK293 Human | 0.4 | 0.5 | 0.0 | 0.0 | 92.1 | 0.0 | 3.9 |
| TP63 17297297 ChIP-ChIP HaCaT Human | 9.8 | 2.4 | 0.0 | 0.0 | 60.4 | 0.0 | 13.7 |
| CDX2 20551321 ChIP-Seq CACO-2 Human | 5.3 | 2.5 | 17.3 | 0.0 | 36.3 | 0.0 | 4.8 |
| PPAR 26484153 Chip-Seq NCI-H1993 Human | 8.5 | 9.6 | 3.9 | 0.0 | 23.7 | 17.5 | 3.2 |
| CREB1 26743006 Chip-Seq LNCaP Human | 2.2 | 3.7 | 16.7 | 6.6 | 27.7 | 20.7 | 6.4 |
| ESR1 20079471 ChIP-ChIP T-47D Human | 10.4 | 2.7 | 13.0 | 0.0 | 26.1 | 42.6 | 11.8 |
| PPARD 23208498 ChIP-Seq MDA-MB-231 Human | 11.5 | 2.3 | 0.0 | 0.0 | 6.2 | 60.4 | 4.5 |
| IRF1 19129219 ChIP-ChIP H3396 Human | 8.2 | 1.5 | 0.0 | 0.0 | 6.2 | 48.6 | 0.9 |
| E2F1 20622854 ChIP-Seq HELA Human | 0.9 | 1.0 | 3.9 | 0.3 | 19.5 | 44.5 | 4.7 |
| IRF1 21803131 ChIP-Seq MONOCYTES Human | 0.4 | 0.7 | 6.3 | 9.2 | 2.4 | 22.3 | 1.6 |
| GATA6 25053715 ChIP-Seq YYC3 Human | 2.2 | 12.2 | 3.9 | 0.3 | 19.5 | 20.7 | 4.7 |
| KLF6 26769127 Chip-Seq PDAC-Cell line Human | 6.1 | 6.2 | 8.9 | 0.3 | 19.5 | 20.7 | 6.4 |
| BCOR 27268052 Chip-Seq Bcells Human | 0.4 | 1.0 | 0.2 | 0.3 | 4.6 | 20.7 | 10.8 |
| AR 21909140 ChIP-Seq LNCAP Human | 0.1 | 6.8 | 8.0 | 11.5 | 3.1 | 0.0 | 24.7 |

TABLE 9
Enrichr enrichment analysis of trajectory associate genes

| | | Enrichment |
|---|---|---|
| | Gene set enrichment | score |
| Trajta1A | CREM 20920259 ChIP-Seq GC1-SPG Mouse | 97.63 |
| associated | FOXM1 25889361 ChIP-Seg OE33 AND U2OS Human | 99.41 |
| genes | E2F4 21247883 ChIP-Seg LYMPHOBLASTOID Human | 67.94 |
| | RBPJ 21746931 ChIP-Seq IB4 Human | 72.63 |
| | KDM5B 21448134 ChIP-Seq MESCs Mouse | 62.79 |
| | GATA6 21074721 ChIP-Seq CACO-2 Human | 68.38 |
| | GATA 21074721 ChIP-Seq CACO-2 Mouse | 64.29 |
| | PHF8 20622853 ChIP-Seq HELA Human | 60.34 |
| | CDX2 21074721 ChIP-Seq CACO-2 Mouse | 60.34 |
| | PCGF4 22325352 ChIP-Seg 293T-Rex Human | 59.06 |
| | ETV1 20927104 ChIP-Seq GIST48 Human | 57.80 |
| | P300 27058665 Chip-Seq ZR-75-30cells Human | 57.80 |
| | HNFA 21074721 ChIP-Seg CACO-2 Human | 55.32 |
| | CBX2 22325352 ChIP-Seq 293T-Rex Human | 55.32 |
| | EKLF 21900194 ChIP-Seq ERYTHROCYTE Mouse | 61.60 |
| | NFYB 21822215 ChIP-Seq K562 Human | 49.42 |
| | NFYA 21822215 ChIP-Seq K562 Human | 44.98 |
| | KLF4 18555785 ChIP-Seq MESCs Mouse | 40.41 |
| | KLF4 26769127 Chip-Seq PDAC-Cell line Human | 41.82 |
| | FOXM1 23109430 ChIP-Seq U2OS Human | 73.36 |
| | KLF6 26769127 Chip-Seq PDAC-Cell line Human | 39.79 |
| | XRN2 22483619 ChIP-Seq HELA Human | 41.45 |
| | CREB1 23762244 ChIP-Seg HIPPOCAMPUS Rat | 36.76 |
| | MYCN 18555785 ChIP-Seq MESCs Mouse | 36.49 |
| | TTF2 22483619 ChIP-Seq HELA Human | 36.59 |
| | FOXP1 21924763 ChIP-Seq HESCs Human | 29.67 |
| | TBL1 22424771 ChIP-Seq 293T Human | 31.40 |
| | PRDM5 23873026 ChIP-Seq MEFs Mouse | 35.85 |
| | EZF4 17652178 ChIP-ChIP JURKAT Human | 34.37 |
| | CHD1 26751641 Chip-Seq LNCaP Human | 29.69 |
| | ELF3 26769127 Chip-Seq PDAC-Cell line Human | 29.69 |
| | EZF1 21310950 ChIP-Seq MCF-7 Human | 32.95 |
| | GABP 19822575 ChIP-Seq HepG2 Human | 27.34 |
| | KDM5A 27292631 Chip-Seq BREAST Human | 31.70 |
| | DACH1 20351289 ChIP-Seq MDA-MB-231 Human | 26.43 |
| | CTCF 27219007 Chip-Seq ERYTHROID Human | 24.93 |
| | NELFA 20434984 ChIP-Seq ESCs Mouse | 24.18 |
| | ZFX 18555785 ChIP-Seq MESCs Mouse | 22.26 |
| | MYC 19030024 ChIP-ChIP MESCs Mouse | 21.48 |
| | KDM2B 26808549 Chip-Seq DND41 Human | 23.45 |
| | KDM2B 26808549 Chip-Seq SIL-ALL Human | 23.45 |
| | FOXM1 26100407 CHIP-SEQ Hek293 flp-in Human | 22.88 |
| | FOXO3 22982991 ChIP-Seq MACROPHAGES Mouse | 22.03 |
| | KDMZB 26808549 Chip-Seq JURKAT Human | 22.03 |
| | FOXA1 26743006 Chip-Seq LNCaP-abl Human | 20.66 |
| | KDM2B 26808549 Chip-Seq HPB-ALL Human | 20.66 |
| | VDR 23849224 ChIP-Seq CD4+ Human | 19.21 |
| | ERG 20887958 ChIP-Seq HPC-7 Mouse | 18.62 |
| | UBF1_2 26484160 Chip-Seq HMEC-DERIVED Human | 18.08 |
| | FOXA1 26769127 Chip-Seq PDAC-Cell line Human | 18.08 |
| | KDM2B 26808549 Chip-Seq K562 Human: | 16.87 |
| | ETS1 20019798 ChIP-Seq JURKAT Human | 17.07 |
| | RACK7 27058665 Chip-Seq MCF-7 Human | 16.29 |
| | SIN3B 21632747 ChIP-Seq MESCs Mouse | 14.03 |
| | TAL1 20887958 ChIP-Seq HPC-7 Mouse | 15.12 |
| | E2F7 22180533 ChIP-Seq HELA Human | 37.21 |
| | OCT4 18692474 ChIP-Seq MEFs Mouse | 13.40 |
| | CREB1 20920259 ChIP-Seq GC1-SPG Mouse | 12.24 |
| | TRIM28 19339689 ChIP-ChIP MESCs Mouse | 11.73 |
| | MITF 21258399 ChIP-Seq MELANOMA Human | 11.01 |
| | JARID1A 20064375 ChIP-Seq MESCs Mouse | 11.96 |
| | RUNX1 21571218 ChIP-Seq MEGAKARYOCYTES Human | 10.65 |
| | MAF 26560356 Chip-Seq TH2 Human | 11.57 |
| | E2F1 18555785 ChIP-Seq MESCs Mouse | 10.59 |
| | FOXP3 21729870 ChIP-Seq TREG Human | 11.96 |
| | TCFCP2L1 18555785 ChIP-Seq MESCs Mouse | 11.17 |
| | SA1 27219007 Chip-Seq Bcells Human | 10.65 |
| | ELK3 25401928 ChIP-Seq HUVEC Human | 10.21 |
| | MYB 26560356 Chip-Seq TH1 Human | 10.21 |
| | HOXB4 20404135 ChIP-ChIP EML Mouse | 10.01 |
| | CREB1 26743006 Chip-Seq LNCaP Human | 9.78 |
| | DMRT1 23473982 ChIP-Seq TESTES Mouse | 9.65 |
| | BCL3 23251550 ChIP-Seq MUSCLE Mouse | 10.62 |
| | YY1 23942234 ChIP-Seq MYOBLASTS AND | 9.50 |
| | MYOTUBES Mouse | |
| | SFPI1 20887958 ChIP-Seq HPC-7 Mouse | 8.94 |
| | SOX2 18692474 ChIP-Seq MEFs Mouse | 8.87 |
| | NANOG 21062744 ChIP-ChIP HESCs Human | 9.74 |
| | SOX9 26525672 Chip-Seq Limbbuds Mouse | 9.14 |
| | KDM2B 26808549 Chip-Seq SUP-B15 Human | 8.56 |
| Traj1B | FOXM1 23109430 ChIP-Seq U2OS Human | 91.50 |
| associated | FOXM1 25889361 ChIP-Seq OE33 AND U2OS Human | 53.98 |
| genes | CBX2 22325352 ChIP-Seq 293T-Rex Human | 33.65 |
| | NFYB 21822215 ChIP-Seq K562 Human | 29.72 |
| | RBPJ 21746931 ChIP-Seq I84 Human | 29.72 |
| | NFYA 21822215 ChIP-Seq K562 Human | 30.68 |
| | GATA6 21074721 ChIP-Seq CACO-2 Human | 29.72 |
| | CDX2 21074721 ChIP-Seq CACO-2 Mouse | 29.72 |
| | HNFA 21074721 ChIP-Seq CACO-2 Human | 26.97 |
| | KDM2B 26808549 Chip-Seq K562 Human | 26.09 |
| | ETV1 20927104 ChIP-Seq GIST48 Human | 26.09 |
| | PCGF4 22325352 ChIP-Seq 293T-Rex Human | 25.23 |
| | GATA6 21074721 ChIP-Seq CACO-2 Mouse | 25.23 |
| | PHF8 20622853 ChIP-Seq HELA Human | 25.23 |
| | KDM2B 26808549 Chip-Seq JURKAT Human | 21.17 |
| | TBL1 22424771 ChIP-Seq 293T Human | 20.42 |
| | P300 27058665 Chip-Seq ZR-75-30cells Human | 18.95 |
| | KLF4 26769127 Chip-Seq PDAC-Cell line Human | 18.24 |
| | EZF7 22180533 ChIP-Seq HELA Human | 45.53 |
| | FOXM1 26100407 CHIP-SEQ Hek293 flp-in Human | 12.48 |
| | KDM28 26808549 Chip-Seq SIL-ALL Human | 10.99 |
| Traj2 | TTF2 22483619 ChIP-Seq HELA Human | 72.85 |
| associated | EKLF 21900194 ChIP-Seg ERYTHROCYTE Mouse | 62.98 |
| genes | MYC 19079543 ChIP-ChIP MESCs Mouse | 51.91 |
| | XRN2 22483619 ChIP-Seq HELA Human | 49.27 |
| | E2F1 18555785 ChIP-Seq MESCs Mouse | 35.77 |
| | MYC 19030024 ChIP-ChIP MESCs Mouse | 35.88 |
| | MYC 18358816 ChIP-ChIP MESCs Mouse | 34.86 |
| | MYC 18555785 ChIP-Seq MESCs Mouse | 33.13 |
| | NELFA 20434984 ChIP-Seq ESCs Mouse | 25.15 |
| | VDR 23849224 ChIP-Seq CD4+ Human | 23.92 |
| | AR 21909140 ChIP-Seq LNCAP Human | 40.25 |
| | FOXA1 21915096 ChIP-Seq LNCaP-1F5 Human | 22.06 |
| | HOXC9 25013753 ChIP-Seq NEUROBLASTOMA BEZ-C Human | 21.34 |
| | ERG 20887958 ChIP-Seq HPC-7 Mouse | 18.86 |
| | TAL1 20887958 ChIP-Seq HPC-7 Mouse | 17.94 |
| | ETS1 20019798 ChIP-Seq JURKAT Human | 16.90 |
| | BCL3 23251550 ChIP-Seq MUSCLE Mouse | 19.43 |
| | CEBPB 24764292 ChIP-Seq MC3T3 Mouse | 15.79 |
| | DCP1A 22483619 ChIP-Seq HELA Human | 17.67 |
| | CHD1 26751641 Chip-Seq LNCaP Human | 14.24 |
| | AR 21915096 ChIP-Seq LNCaP-1F5 Human | 14.24 |
| | ZFX 18555785 ChIP-Seq MESCs Mouse | 12.57 |
| | CREM 20920259 ChIP-Seq GC1-SPG Mouse | 10.74 |
| | JARID1A 20064375 ChIP-Seq MESCs Mouse | 11.18 |
| Traj3A | CLOCK 20551151 ChIP-Seq 293T Human | 31.42 |
| associated | CHO1 26751641 Chip-Seq LNCaP Human | 31.13 |
| genes | FOXP1 22492998 ChIP-Seq STRATIUM Mouse | 31.05 |
| | ZNF217 24962896 ChIp-Seq MCF-7 Human | 20.56 |
| | ZNF263 19887448 ChIP-Seq K562 Human | 16.31 |
| | FOXA1 25552417 ChIP-Seq VCAP Human | 14.63 |
| | BCL11B 21912641 ChIP-Seq STHDH STRIUM Mouse | 14.14 |
| | PRDM5 23873026 ChIP-Seq MEFs Mouse | 13.82 |
| | NR1H3 23393188 ChIP-Seq ATHEROSCLEROTIC-FOAM Human | 13.01 |
| | ELK3 25401928 ChIP-Seq HUVEC Human | 12.88 |
| | DACH1 20351289 ChIP-Seq MDA-MB-231 Human | 12.87 |
| | CREM 20920259 ChIP-Seq GC1-SPG Mouse | 12.85 |
| | VDR 21846776 ChIP-Seq THP-1 Human | 12.69 |
| | SMAD4 19686287 ChIP-ChIP HaCaT Human | 10.37 |
| Traj3B | ZNF652 21678463 ChIP-ChIP ZR75-1 Human | 94.50 |
| associated | NUCKS1 24931609 ChIP-Seq HEPATOCYTES Mouse | 36.03 |
| genes | FOXP1 22492998 ChIP-Seq STRATIUM Mouse | 33.18 |
| | GATA1 22025678 ChIP-Seq K562 Human | 19.52 |
| | HOXA2 22223247 ChIP-Seq E11.5 EMBRYO Mouse | 19.52 |
| | STAT3 22323479 ChIP-Seq MACROPHAGE Mouse | 19.52 |
| | GATA3 27048872 Chip-Seq THYMUS Human | 18.34 |
| | STAT6 21828071 ChIP-Seq BEAS2B Human | 17.51 |
| | POU3F2 20337985 ChIP-ChIP 501MEL Human | 16.80 |
| | BCL11B 21912641 ChIP-Seq STHDH STRIUM Mouse | 16.35 |
| | CDX2 22108803 ChIP-Seq LS180 Human | 14.51 |
| | TFEB 21752829 ChIP-Seq HELA Human | 13.22 |
| | GABP 17652178 ChIP-ChIP JURKAT Human | 12.34 |
| | CCND1 20090754 ChIP-CHIP RETINA Mouse | 12.23 |
| | NANOG 21062744 ChIP-ChIP HESCs Human | 12.16 |
| | CREB1 23762244 ChIP-Seq HIPPOCAMPUS Rat | 11.56 |
| | CREM 20920259 ChIP-Seg GC1-SPG Mouse | 11.39 |
| | HOXB7 26014856 ChIP-Seq BT474 Human | 11.23 |
| | TAL1 26923725 Chip-Seq HPCs Mouse | 11.23 |
| | MEF2A 21415370 ChIP-Seq HL-1 Mouse | 11.13 |
| | IKZF1 21737484 ChIP-CHIP HCT116 Human | 10.92 |

The systems and methods described herein can be implemented in hardware, software, firmware, or combinations of hardware, software and/or firmware. In some examples, the systems and methods described in this specification may be implemented using a non-transitory computer readable medium storing computer executable instructions that when executed by one or more processors of a computer cause the computer to perform operations. Computer readable media suitable for implementing the systems and methods described in this specification include non-transitory computer-readable media, such as disk memory devices, chip memory devices, programmable logic devices, random access memory (RAM), read only memory (ROM), optical read/write memory, cache memory, magnetic read/write memory, flash memory, and application-specific integrated circuits. In addition, a computer readable medium that implements a system or method described in this specification may be located on a single device or computing platform or may be distributed across multiple devices or computing platforms.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. A method of determining the risk of disease progression of prostate cancer in a subject, the method comprising:
(i) obtaining a biological sample from the subject, said sample comprising prostate cancer cells; and
(ii) determining a castration-resistant prostate cancer (CRPC) evolutionary signature of the prostate cancer cells to generate a score,
wherein a sample having a high score as compared to a control is indicative of aggressive prostate cancer.

2. The method of claim 1, wherein the signature comprises a plurality of genes and the determining comprises measuring the expression levels of the genes.

3. The method of claim 2, wherein the measuring is carried out with quantitative PCR such as rtPCR.

4. The method of claim 2-or claim 3, wherein the signature comprises from 3, 5, 8 or 10 to 20, 25, 30, 35, 40, 45, or 51 of the genes listed in Table 1.

5. The method of claim 4, wherein the signature comprises from 3, 5, 8 or 10 to 20, 25, 30, 35, 40, 45, or 51 of the genes listed as CRPCsig51_old as provided in Table 1.

6. The method of claim 4, wherein the signature comprises from 3, 5, 8 or 10 to 20, 25, 30, 35, 40, 45, or 51 of the genes listed as CRPCsig51_new as provided in Table 1.

7. The method of claim 4, wherein the signature comprises from 3, 4, 5, or 6, to 8, 9, 10, 11, or 12 of the genes listed in Table 4.

8. The method of claim 1, wherein the score is determined by a Youden's index, or maximum specificity and sensitivity.

9. The method of claim 1, wherein the subject has an early-stage prostate cancer diagnosis and is determined to be of intermediate risk.

10. A method of detecting and treating an aggressive prostate cancer in a subject, the method comprising:
(i) obtaining a biological sample from the subject, said sample comprising prostate cancer cells;
(ii) determining a castration-resistant prostate cancer (CRPC) evolutionary signature in the sample to generate a score, in which samples from the subject have a higher score as compared to a control, which higher score is indicative of the aggressive prostate cancer; and then
(iii) administering a prostate cancer treatment to the subject determined to have aggressive prostate cancer.

11. The method of claim 10, wherein the prostate cancer treatment comprises one or more of surgery (e.g. prostatectomy), radiation, and focal treatment (e.g., heat, cold, or laser treatment).

12. The method of claim 10, wherein the signature comprises a plurality of genes and the determining comprises measuring the expression levels of the genes.

13. The method of claim 12, wherein the measuring is carried out with quantitative PCR such as rtPCR.

14. The method of claim 12, wherein the signature comprises from 3, 5, 8 or 10 to 20, 25, 30, 35, 40, 45, or 51 of the genes listed in Table 1.

15. The method of claim 14, wherein the signature comprises from 3, 5, 8 or 10 to 20, 25, 30, 35, 40, 45, or 51 of the genes listed as CRPCsig51_old as provided in Table 1.

16. The method of claim 14, wherein the signature comprises from 3, 5, 8 or 10 to 20, 25, 30, 35, 40, 45, or 51 of the genes listed as CRPCsig51_new as provided in Table 1.

17. The method of claim 14, wherein the signature comprises from 3, 4, 5, or 6, to 8, 9, 10, 11, or 12 of the genes listed in Table 4.

18. The method of claim 10, wherein the score is determined by a Youden's index, or maximum specificity and sensitivity.

19. The method of claim 10, wherein the subject has an early-stage prostate cancer diagnosis and is determined to be of intermediate risk.

20.-23. (canceled)
